(19) United States
(12) Patent Application Publication
(10) Pub. No.: US 2017/0073659 A1
(43) Pub. Date: Mar. 16, 2017

(54) NOVEL LYSYL TRNA SYNTHETASE FRAGMENT AND MICROVESICLES COMPRISING SAME

(71) Applicant: Medicinal Bioconvergence Research Center, Gyeonggi-do (KR)

(72) Inventors: Sunghoon Kim, Seoul (KR); Min-Chul Park, Gyeonggi-do (KR); Sang-Bum Kim, Seoul (KR)

(21) Appl. No.: 15/361,698

(22) Filed: Nov. 28, 2016

Related U.S. Application Data
(63) Continuation of application No. PCT/KR2015/005371, filed on May 28, 2015.

(30) Foreign Application Priority Data
May 28, 2014 (KR) 1020140064612

(51) Int. Cl. C12N 9/00

(52) U.S. Cl. CPC C12N 9/93; C12Y 601/01006

(57) ABSTRACT

The present invention relates to: a lysyl tRNA synthetase (KRS) fragment which comprises an amino acid sequence represented by SEQ ID NO: 1 and is secreted from cancer cells; microvesicles comprising the KRS fragment; and a method for providing information necessary for cancer diagnosis and screening a cancer metastasis inhibiting agent using the same. The present invention can be favorably used in the development of a diagnostic kit for providing information necessary for cancer diagnosis or the development of a cancer metastasis inhibiting agent, and thus is highly industrially applicable.

This is a Continuation of PCT Application No. PCT/KR2015/005371, filed May 28, 2015, which claims the benefit of Korean Application No. 10-2014-0064612 filed on 28 May 2014, the contents of which are incorporated fully by reference herein.

### TECHNICAL FIELD

The present invention relates to a novel lysyl tRNA synthetase fragment and microvesicles containing the same and, more specifically, to a lysyl tRNA synthetase (KRS) fragment comprising the amino acid sequence represented by SEQ ID NO: 1 and being secreted from cancer cells, microvesicles containing the KRS fragment, and methods for providing information necessary for the diagnosis of cancer and screening a cancer metastasis inhibiting agent using the same.

### BACKGROUND ART

A cancer (or tumor) is developed by uncontrollable disordered abnormal cell proliferation. Especially, if this tumor shows destructive growth, invasiveness, and metastasis, it is regarded as a malignant cancer. Invasiveness is a character to infiltrate or destroy surrounding tissues, and in particular, a basal layer forming a boundary of tissues is destroyed thereby, resulting in the local spread and sometimes inflow of a tumor through the circulatory system. Metastasis means the spread of tumor cells from their originated place to other areas through lymphatic or blood vessels. In a broad sense, metastasis also means the direction elongation and migration of tumor cells through serous body cavity or other space. In order to treat such cancer, the development of cancer diagnosis with high accuracy and specificity prior to the treatment is very important, while customized diagnosis and treatment through the prediction of the prognosis of cancer (and metastasis thereof) is further requested.

Meanwhile, cytokines are secreted from many cells, especially immune cells. In addition, cytokines transfer intercellular signals for regulating the proliferation, differentiation, migration, and apoptosis of cells. Cytokines have no function inside cells, and thus must be secreted for exerting their effects. Most cytokines include signal peptide sequences directing toward the endoplasmic reticulum at their amino terminus. The COP II complex transfers cytokines from the endoplasmic reticulum to golgi bodies, and then the cytokines are secreted extracellularly. That is, classical cytokines present inside cells (for example, TNF-alpha, TGF-beta, and IL1-beta) have no intracellular functions, while being secreted extracellularly via ER-golgi using the signal peptide sequences thereof to function outside cells.

Non-classical cytokines function even intracellularly, and thus have no signal peptide sequences at the amino terminus thereof. For this reason, the non-classical cytokines are secreted through non-universal secretion pathways such as secretion-mediated carriers, microvesicle shedding, exosome emission, and secretory lysosomes. Out of the non-classical secretion pathways, the exosomes transfer intercellular molecules, such as intracellular proteins, mRNA, and micro RNA. The exosomes are membrane-derived microvesicles with a diameter of 30-100 nm. The exosomes originate from the inside of multi-vesicular bodies (MVBs), and are secreted extracellularly in a mixed form with cell membrane-derived endosomes. Out of various types of cells, cancer and immune cells secrete exosomes for mediating intercellular interactions.

KRS belongs to aminoacyl-tRNA synthetases (ARSs) that ligate their cognate amino acids and tRNAs for protein synthesis. These ancient enzymes show pleiotropic functions in addition to their catalytic activities (Park, S. G., Ewalt, K. L. & Kim, S. Functional expansion of aminoacyl-tRNA synthetases and their interacting factors: new perspectives on housekeepers. Trends Biochem. Sci. 30, 569-574 (2005)). Besides, several mammalian ARSs including KRS form macromolecular complexes which serve as molecular reservoirs (Ray, P. S., Arif, A. & Fox, P., Macromolecular complexes as depots for releasable regulatory proteins, Trends Biochem. Sci. 32, 158-164 (2007)), to control multiple functions of the component proteins (Lee, S. W., Cho, B. H., Park, S. G. & Kim, S. Aminoacyl-tRNA synthetase complexes: beyond translation. J. Cell. Sci. 117, 3725-3734 (2004); Han, J. M., Kim, J. Y. & Kim, S., Molecular network and functional implications of macromolecular tRNA synthetase complex., Biochem. Biophys. Res. Commun. 303, 985-993 (2003)).

It has been known that KRS proteins are secreted from cancer cells to increase TNF-alpha secretion through macrophage and macrophage migration, causing inflammation responses. It has been known that KRS is secreted at the serum starvation, while the secretion of KRS is increased upon the simultaneous treatment with TNF-alpha. Little is known about how the KRS is secreted.
[Non-patent document 1] K. Choe, Y. Hwang, H. Seo, and P. Kim, “In vivo high spatiotemporal resolution visualization of circulating T lymphocytes in high endothelial venules of lymph nodes.,” J. Biomed. Opt., vol. 18, no. 3, p. 036005, March 2013.
[Non-patent document 2] N. Faust, F. Varas, L. M. Kelly, S. Heck, and T. Graf, “Insertion of enhanced green fluorescent protein into the lysozyme gene creates mice with green fluorescent granulocytes and macrophages,” vol. 96, no. 2, pp. 719726, 2000.

### DETAILED DESCRIPTION OF THE INVENTION

### Technical Problem

The present inventors, while researching KRS secretion mechanisms, established a new fact that, for extracellular secretion, KRS is necessarily cleaved by caspase-8, and a KRS fragment generated by the cleavage is secreted extracellularly through exosomes, and verified uses of the KRS fragment and microvesicles (especially, exosomes) containing the same for diagnosing cancer and screening a cancer metastasis inhibiting agent, and then completed the present invention.

Therefore, an aspect of the present invention is to provide a lysyl tRNA synthetase fragment including the amino acid sequence represented by SEQ ID NO: 1 and being secreted from cancer cells.

Another aspect of the present invention is to provide microvesicles containing the lysyl tRNA synthetase fragment and being secreted from cancer cells.

Another aspect of the present invention is to provide a method for providing information necessary for diagnosis of cancer, the method including: (a) isolating microvesicles from a biological sample taken from a suspected cancer subject; (b) disrupting the microvesicles in step (a) to measure the level of the lysyl tRNA synthetase fragment of SEQ ID NO: 1 or the expression level of a gene encoding the fragment; and (c) comparing the level of the fragment or the expression level of the gene encoding the fragment with the level of the fragment or the expression level of the gene encoding the fragment in a normal control sample.

Still another aspect of the present invention is to provide a method for screening a cancer metastasis inhibiting agent, the method comprising: (a) bringing a lysyl tRNA synthetase (KRS) or KRS fragment comprising the C-terminal region thereof, syntenin, and a test agent into contact with one another; (B) measuring the change in the binding level of the KRS or fragment thereof and syntenin; and (C) bringing the test agent, which is determined to change the binding level of the KRS or fragment thereof and syntenin, into contact with cancer cells, to evaluate whether microvesicles containing the lysyl tRNA synthetase fragment of SEQ ID NO: 1 are secreted from the cancer cells.

### Technical Solution

In accordance with an aspect of the present invention, there is provided a lysyl tRNA synthetase fragment comprising the amino acid sequence represented by SEQ ID NO: 1, and being secreted from cancer cells.

In accordance with another aspect of the present invention, there are provided microvesicles containing the lysyl tRNA synthetase fragment of SEQ ID NO: 1 and being secreted from cancer cells.

In accordance with another aspect of the present invention, there is provided a method for providing information necessary for diagnosis of cancer, the method including: (a) isolating microvesicles from a biological sample taken from a suspected cancer subject; (b) disrupting the microvesicles in step (a) to measure the level of the lysyl tRNA synthetase fragment of SEQ ID NO: 1 or the expression level of a gene encoding the fragment; and (c) comparing the level of the fragment or the expression level of the gene encoding the fragment with the level of the fragment or the expression level of the gene encoding the fragment in a normal control sample.

In accordance with another aspect of the present invention, there is provided a method for screening a cancer metastasis inhibiting agent, the method comprising: (a) bringing a lysyl tRNA synthetase (KRS) or a KRS fragment comprising the C-terminal region thereof, syntenin, and a test agent into contact with one another; (B) measuring a change in the binding level of the KRS or fragment thereof and syntenin; and (C) bringing the test agent, which is determined to change the binding level of the KRS or fragment thereof and syntenin, into contact with cancer cells, to evaluate whether microvesicles containing the lysyl tRNA synthetase fragment of SEQ ID NO: 1 are secreted from the cancer cells.

Hereinafter, the present invention will be described in detail.

### DEFINITIONS

Unless defined otherwise, all technical and scientific terms used herein have the same meanings as are commonly understood by a person skilled in the art. The following reference documents provide one of the skills having general definitions of many terms used herein. Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY. Also, the following definitions are provided to help readers for the implementation of the present invention.

As used herein, the term “protein” is used interchangeably with the term “polypeptide” or “peptide”, and refers to a polymer of amino acid residues, as typically found in proteins in nature.

As used herein, the term “nucleic acid” or “polynucleotide” refers to a single- or double-stranded deoxyribonucleotide or ribonucleotide. Unless otherwise limited, the term encompasses known analogs of natural nucleotides that hybridize to nucleic acids in a manner similar to naturally-occurring nucleotides.

An aspect of the present invention provides a lysyl tRNA synthetase fragment (KRS fragment) including the amino acid sequence of SEQ ID NO: 1 and being secreted from cancer cells.

Here, the term “KRS” refers to a full-length polypeptide known in the art as a lysyl tRNA synthetase in the art. The specific amino acid sequence of KRS is not particularly limited as long as it is known as a lysyl tRNA synthetase in the art, but examples thereof include SEQ ID NO: 2 (Genbank Accession No. NP_005539.1), SEQ ID NO: 3 (Genbank Accession No. NP_001123561.1), and the like. Herein, KRS may preferably mean a polypeptide composed of the amino acid sequence represented by SEQ ID NO: 2 (Genbank Accession No. NP_005539.1).

As used herein, the term “KRS fragment” refers to a partial fragment of the KRS polypeptide full-length sequence, and the KRS fragment of the present invention preferably means the fragment represented by SEQ ID NO: 1, in which 1st to 12th amino acids from the N-terminus of the known human KRS (Genbank Accession No. NP_005539.1) are deleted. The present inventors have first established the fact that the full-length sequence of a polypeptide known as the KRS protein is not secreted from cancer cells, but KRS is engineered into a KRS fragment (SEQ ID NO: 1) with a partial region cleaved, and here, microvesicles, such as exosomes, are used as a secretion unit, and this process is indispensable when the KRS-derived component involved in the creation of microenvironments of cancer metastasis shows their activity. This fact is well described in following Examples of the present specification.

The present invention provides a lysyl tRNA synthetase fragment composed of the amino acid sequence of SEQ ID NO: 1 and being secreted from cancer cells, while the KRS fragment according to the present invention may include functional equivalents thereof.

The term “functional equivalent” refers to a polypeptide having sequence homology (that is, identity) of at least 70%, preferably at least 80%, and more preferably at least 90%, for example, 70%, 71%, 72%, 73%, 740, 75%, 760, 77%, 78%, 79%, 800, 81%, 82%, 83%, 84%, 85%, 86%, 87%, 88%, 89%, 90%, 91%, 92%, 93%, 94%, 95%, 96%, 97%, 98%, 99%, and 100% amino acid sequence homology, that exhibit substantially identical physiological activity to the polypeptide represented by SEQ ID NO: 1. The functional equivalent may include, for example peptides produced as a result of addition, substitution, or deletion of some amino acids of the amino acid sequence of SEQ ID NO: 1. Herein, substitutions of the amino acids are preferably conservative substitutions. Examples of conservative substitutions of naturally occurring amino acids are as follows: aliphatic amino acids (Gly, Ala, Pro), hydrophobic amino acids (Ile, Leu, Val), aromatic amino acids (Phe, Tyr, Trp), acidic amino acids (Asp, Glu), basic amino acids (His, Lys, Arg, Gln, Asn), and sulfur-containing amino acids (Cys, Met). Furthermore, the functional equivalent also includes variants with some amino acid deletions on the amino acid sequence of the KRS fragment polypeptide. The deletions or substitutions of amino acids are preferably located at regions that are not directly involved in the physiological activity of the polypeptide of the present invention. The deletions of the amino acids are preferably located at regions that are not directly involved in the physiological activity of the KRS fragment polypeptide. In addition, the functional equivalent also includes variants with the addition of several amino acids at either terminal ends or inside the sequence of the KRS fragment polypeptide. Moreover, the functional equivalent of the present invention also includes polypeptide derivatives that have some modification of certain chemical structure of the polypeptide according to the present invention, while maintaining the basic backbone and physiological activity thereof. Examples of the modification include structural modifications for changing the stability, storability, volatility, or solubility of the polypeptide of the present invention.

The sequence identity or homology is defined herein as the percentage of amino acid residues of a candidate sequence over the amino acid sequence of the KRS fragment (SEQ ID NO: 1), after aligning the respective sequences and introducing gaps. If necessary, in order to achieve a maximum percentage of sequence identity, any conservative substitution as a part of the sequence identity is not considered. In addition, none of N-terminus, C-terminus, or internal extensions, deletions, or insertions of the amino acid sequence of the KRS fragment shall be construed as affecting sequence identity or homology. Further, the sequence identity may be determined by a general standard method that is commonly used to compare similar residues of amino acid sequences of two polypeptides. Using a computer program, such as BLAST or FASTA, two polypeptides are aligned for optimal matching of their respective amino acids (either along the full-length sequence of one or both sequences or along a predetermined portion of one or both sequences). The computer program provides a default opening penalty and a default gap penalty, and provides a scoring matrix, such as PAM 250 (a standard scoring matrix; see Dayhoff et al., in Atlas of Protein Sequence and Structure, vol 5, supp 3, 1978) that can be used in conjunction with the computer program. For example, the percent identity may be calculated as follows. The total number of identical matches is multiplied by 100 and then divided by the sum of the length of the longer sequence within the matched span and the number of gaps introduced into the longer sequences in order to align the two sequences.

The KRS fragment according to the present invention may be extracted from the nature or may be constructed by a genetic engineering method. For example, a nucleic acid (e.g., the polynucleotide sequence of SEQ ID NO: 5) encoding the KRS fragment or the functional equivalent thereof is constructed by a conventional method. The nucleic acid may be constructed by PCR amplification using appropriate primers. Alternatively, the DNA sequence may be synthesized by a standard method known in the art, for example, using an automatic DNA synthesizer (commercially available from Biosearch or Applied Biosystems). The constructed nucleic acid is inserted into a vector including at least one expression control sequence (e.g., promoter, enhancer, etc.) that is operatively linked to the DNA sequence so as to control the expression of the DNA molecule, while host cells are transformed with the resulting recombinant expression vector. The formed transformant is incubated in media under conditions suitable to express the nucleic acid, and a substantially pure polypeptide, which is expressed by the nucleic acid, is collected from the culture product. The collection may be performed using a method known in the art (e.g., chromatography). Herein, the term “substantially pure polypeptide” means that the polypeptide according to the present invention does not substantially contain any other protein derived from host cells. For the genetic engineering method for synthesizing the polypeptide of the present invention, the following literatures may be referred to: Maniatis et al., Molecular Cloning; A laboratory Manual, Cold Spring Harbor laboratory, 1982; Sambrook et al., Molecular Cloning: A Laboratory Manual, Cold Spring Harbor Press, N.Y., Second (1998) and Third (2000) Editions Gene Expression Technology, Method in Enzymology, Genetics and Molecular Biology, Method in Enzymology, Guthrie & Fink (eds.), Academic Press, San Diego, Calif., 1991; and Hitzeman et al., J. Biol. Chem., 255:12073-12080, 1990.

In addition, the polypeptide of the present invention may be easily prepared by a chemical synthesis known in the art (Creighton, Proteins; Structures and Molecular Principles, W. H. Freeman and Co., NY, 1983). Representative examples thereof include, but are not limited to, liquid or solid phase synthesis, fragment condensation, F-MOC or T-BOC chemical method (Chemical Approaches to the Synthesis of Peptides and Proteins, Williams et al., Eds., CRC Press, Boca Raton Fla., 1997; A Practical Approach, Athert on & Sheppard, Eds., IRL Press, Oxford, England, 1989).

The present invention provides microvesicles containing the lysyl tRNA synthetase fragment and being secreted from cancer cells.

As used herein, the term “microvesicles” refers to vesicular particles, each of which has the inside and the outside parts differentiated by a lipid bilayer composed of cellular membrane components, contains cellular membrane lipids, proteins, nucleic acids, and other cellular components, and has a smaller size than the original cell thereof.

The microvesicles that are used as an extracellular secretion means by the KRS fragment of SEQ ID NO: 1 may be any one selected from the group consisting of exosomes, exosome-like microvesicles, epididimosomes, argosomes, promininosomes, prostasomes, dexosomes, texosomes, archeosomes, and oncosomes. Preferably, the microvesicles in the present invention may be exosomes or exosome-like microvesicles, and more preferably exosomes.

The microvesicles of the present invention may have a diameter of, preferably, 10-1000 nm, more preferably 10-200 nm, and most preferably 50-150 nm.

The KRS fragment and the microvesicles containing the same of the present invention are secreted from cancer cells, wherein the cancer may be at least one selected from the group consisting of breast cancer, colorectal cancer, lung cancer, small cell lung cancer, gastric cancer, liver cancer, blood cancer, bone cancer, pancreatic cancer, skin cancer, head or neck cancer, cutaneous or intraocular melanoma, uterine cancer, ovarian cancer, rectal cancer, anal cancer, colon cancer, fallopian tube carcinoma, endometrial carcinoma, cervical cancer, vaginal cancer, vulvar carcinoma, Hodgkin's disease, esophageal cancer, small intestine cancer, endocrine cancer, thyroid cancer, parathyroid carcinoma, adrenal cancer, soft tissue sarcoma, urethral cancer, penile cancer, prostate cancer, chronic or acute leukemia, lymphocyte lymphoma, bladder cancer, kidney cancer, ureter cancer, renal cell carcinoma, renal pelvic carcinoma, CNS tumor, primary CNS lymphoma, spinal cord tumor, brain stem glioma, and pituitary adenoma, but is not limited thereto.

The microvesicles of the present invention are naturally secreted from in vivo cancer cells, but, in order to obtain the microvesicles of the present invention in large quantities, the secretion of the microvesicles may be induced in vitro through the preparation method including the following steps: (1) subjecting cancer cells to starvation stress; and (2) collecting microvesicles secreted in the cancer cells in above step.

In step (1), the starvation stress is applied to cancer cells to promote the generation of the KRS fragment of SEQ ID NO: 1 in cells and activate the secretion mechanism thereof.

A method for applying starvation stress to cells is known in the art, and for example, cancer cells may be incubated in the serum-free media. The types of cancer that can secret the microvesicles of the present invention are described as above.

In addition, in step (1), a process of treating the cells with TNF-alpha in addition to the starvation stress may be further performed.

In step (2), only microvesicles that are generated from the cells in step (1) and secreted extracellularly are isolated and collected. In step (1), the cell culture medium, in which the cells are incubated with starvation stress, is collected, and structure (particle) fractions with a particular size and/or density assumed to contain the KRS fragment of the present invention are collected from the cell culture medium.

A method of separating and obtaining only particles with desired size and/or density from a mixture is well known in the art, and examples of such a method include density gradient, centrifugation (e.g., ultracentrifugation, density gradient centrifugation, etc.), filtration (e.g., a method using a filter with a particular diameter, etc.), dialysis, and free-flow electrophoresis. Products with desired particle sizes may be obtained by repeatedly performing at least one of the several methods above several times.

In one embodiment, in step (2), it is preferable to obtain microvesicles with a diameter of 10-1000 nm through the foregoing method. More preferably, microvesicles having a diameter of 10-200 nm may be obtained, and most preferably, microvesicles having a diameter of 50-150 nm may be obtained.

In another embodiment, in step (2), it is preferable to obtain microvesicles with a density of 1.09-1.19 g/ml through the foregoing method. Most preferably, microvesicles with a density of 1.09-1.18 g/ml may be obtained.

The KRS fragment represented by SEQ ID NO: 1 of the present invention is specifically secreted extracellularly from cancer cells in the form of microvesicles (especially, exosomes), and thus may be used as a diagnostic marker of cancer in vivo. Moreover, the microvesicles of the present invention, as a natural construct from cancer cells, is a carrier for secreting the KRS fragment of SEQ ID NO; 1 out of cells, and promotes the recruitment of peripheral macrophages and neutrophils (macrophages/neutrophils) and the secretion of cancer metastasis-related cytokines in connection with microenvironments of cancer, thereby creating cancer metastasis environments around cancer tissues. Therefore, the microvesicles of the present invention can function as a biomarker for diagnosis of cancer or cancer metastasis. Accordingly, the present invention provides a marker composition for the diagnosis of cancer or cancer metastasis, the marker composition containing the KRS fragment composed of the amino acid sequence of SEQ ID NO: 1 or microvesicles containing the KRS fragment.

As used herein, the term “diagnosis” encompasses all types of analysis used to determine or derive the prediction or risk of disease incidence.

As used herein, the term “cancer diagnostic marker” refers to a material that is expressed in cancer tissues and cells and is used to verify the occurrence of cancer by checking the expression thereof, and preferably, means an organic biomolecule, such as protein or mRNA, showing a significant difference between normal tissues and cancer tissues. As used herein, the term “cancer metastasis diagnostic marker” refers to a material that is expressed in cancer cells showing symptoms of metastasis and is used to verify the possibility of cancer metastasis by checking the expression thereof, and preferably means an organic biomolecule, such as protein or mRNA, showing a significant difference between normal tissues and cancer tissues. Considering the purpose of the present invention, the cancer diagnostic marker or cancer metastasis diagnostic marker is the KRS fragment of SEQ ID NO: 1 specifically expressed in only various cancer tissues and cells or a microvesicles containing the same, and can diagnose cancer by investigating whether the KRS fragment represented by SEQ ID NO: 1 or the microvesicles containing the same is secreted extracellularly.

A conventional general method for diagnosing cancer is a biopsy wherein a predetermined portion of a tissue, supposed to have an occurrence of cancer at an initial metastatic state, is taken out and observed. However, it is difficult to determine the accurate location of biopsy. Moreover, the conventional method has a risk of causing additional damages and secondary symptoms (that is, complications) in a subject, depending on the extent of invasion at the time of tissue sampling. That is, most of the biological markers maintain the expression state thereof in cells, often require the invasion for taking the corresponding diseased tissues, and the invasive sampling of tissues has a risk of causing or aggravating additional pathological conditions to a subject.

However, the marker of the present invention is processed and secreted through a particular procedure from cancer cells, and thus, when the use of the marker for diagnosing the existence of cancer and the possibility of cancer metastasis allows the diagnosis of cancer (or possibility of cancer metastasis), even without directly taking tissues from the cancer-afflicted area like in the conventional art, through a liquid biopsy or the like, which is a less invasive method. The liquid biopsy is a biopsy in which the body fluid is used instead of living solid tissues of tumor patients. The liquid biopsy provides simpler sampling than an existing biopsy, and is known to cause less pain to the patient with a lower risk of infection.

Furthermore, the present invention provides a composition for diagnosing cancer, the composition comprising a material (reagent) for detecting the presence of the marker of the present invention (i.e., the KRS represented by SEQ ID NO: 1 or microvesicles containing the same), and the amount thereof, pattern thereof, or both. The material for detecting the presence of the marker, and the amount thereof, pattern thereof, or both may be an antibody specific to the marker of the present invention.

In an embodiment of the present invention, when the KRS fragment represented by SEQ ID NO: 1 is used as a marker, the detection of the presence of the marker, and the amount thereof, the pattern thereof, or both may include detection at the level of a protein or detection at the level of a gene encoding the marker, but herein, the detection is preferably a detection at the level of a protein. Here, the reagent detectable at the level of a protein includes monoclonal antibodies, polyclonal antibodies, substrates, aptamers, receptors, ligands or cofactors, or detecting reagents for mass spectroscope, but is not limited thereto.

The material (reagent) used in the detection may be prepared and selected on the basis of the preparation of the KRS fragment represented by SEQ ID NO: 1 or the microvesicles containing the same by the foregoing method and the analysis thereof.

Furthermore, the present invention provides a kit for the diagnosis of cancer, the kit comprising reagents for detecting the marker of the present invention, and an apparatus for analysis of the marker or a computer having an algorithm embedded therein.

In an embodiment, the method for providing information necessary for diagnosing cancer (or the possibility of cancer metastasis) using the marker of the present invention may be performed by comprising the following steps:

(a) isolating microvesicles from a biological sample taken from a suspected cancer subject;

(b) disrupting the microvesicles in step (a) to measure the level of the lysyl tRNA synthetase fragment of SEQ ID NO: 1 or the expression level of a gene encoding the fragment; and

(c) comparing the level of the fragment or the expression level of the gene encoding the fragment with the level of the fragment or the expression level of the gene encoding the fragment in a normal control sample.

Hereinafter, respective steps will be described.

In step (a), microvesicles are isolated from a biological sample taken from a suspected cancer subject.

As used herein, the term “subject” refers to an animal, which is the subject of cancer diagnosis, and preferably a mammal, especially an animal including a human. The subject may be a patient in need of treatment.

The biological sample is isolated and obtained from a suspected cancer subject, and the kind of the biological sample is not particularly limited as long as the sample can be used to obtain microvesicles secreted from cells. Examples of the biological sample may include tissues of cancer, cells, whole blood, blood plasma, serum, saliva, ocular fluid, cerebrospinal fluid, sweat, urine, milk, ascites fluid, synovial fluid, peritoneal fluid, lymph fluid, and the like, but are not limited thereto. Most preferably, the biological sample may be urine, blood, serum, or lymph fluid. The sample may be pre-treated prior to the use for detection. For example, the sample may be pre-treated by filtration, distillation, extraction, concentration, interference ingredient deactivation, reagent addition, and the like.

In step (a), desired microvesicles are isolated from the sample, and here, methods for separating and obtaining only particles with desired size and/or density from the mixture are well known in the art. Examples of such a method include density gradient (e.g., density gradient by ficoll, glycerol, sucrose, or OptiPrep™), centrifugation (e.g., ultracentrifugation, density gradient centrifugation, etc.), filtration (e.g., a method using a filter with a particular diameter, such as gel filtration or ultrafiltration), dialysis, and free-flow electrophoresis. The products with desired particle sizes may be obtained by repeatedly performing at least one of the several methods several times.

In an embodiment, in step (a), it is preferable to obtain microvesicles with a diameter of 10-1000 nm through the foregoing method. More preferably, microvesicles having a diameter of 10-200 nm may be obtained, and most preferably, microvesicles having a diameter of 50-150 nm may be obtained.

In another embodiment, in step (a), it is preferable to obtain microvesicles with a density of 1.09-1.19 g/ml through the foregoing method. Most preferably, microvesicles with a density of 1.09-1.18 g/ml may be obtained.

In step (b), the microvesicles isolated and obtained in step (a) is crushed to measure the level of the lysyl tRNA synthetase fragment of SEQ ID NO: 1 or the expression level of a gene encoding the fragment. In step (c), the level of the KRS fragment or the expression level of the gene encoding the KRS fragment is compared with the level of the KRS fragment or the expression level of the gene encoding the KRS fragment in a normal control sample.

It has been described above that microvesicles (especially exosomes) are used when cancer cells secrete KRS (fragment) extracellularly. Therefore, if the KRS fragment of SEQ ID NO: 1 is detected at a high level (especially, compared with the sample of the normal control group) in the microvesicles isolated from the biological samples obtained from the subject, there is a high possibility that the subject undergoes the state of cancer occurrence or progression.

The detection of the level of the KRS fragment of SEQ ID NO: 1 may be conducted through various immunoassays known in the art. The level of the KRS fragment and the quantitative change thereof may be detected by using radioactive immunoassay, radioactive immunoprecipitation, immunoprecipitation, enzyme-linked immunosorbent assay (ELISA), captured-ELISA, inhibition or competition analysis, and sandwich assay, Ouchterlony immuno diffusion, rocket immunoelectrophoresis, tissue immunostaining, immunoprecipitation assay, complement fixation assay, fluorescence activated cell sorter (FACS), western blotting, and a protein chip, but are not limited thereto.

In addition, the expression level of the gene encoding the KRS fragment may be detected through various methods known in the art. For example, the mRNA expression level may be detected by using RT-PCR (Sambrook et al., Molecular Cloning. A Laboratory Manual, 3rd ed. Cold Spring Harbor Press (2001)), competitive RT-PCR, real-time RT-PCR, RNase protection assay (RPA), northern blotting (Peter B. Kaufma et al., Molecular and Cellular Methods in Biology and Medicine, 102-108, CRC press), hybridization using cDNA microarrays (Sambrook et al., Molecular Cloning. A Laboratory Manual, 3rd ed. Cold Spring Harbor Press (2001)), or in situ hybridization (Sambrook et al., Molecular Cloning. A Laboratory Manual, 3rd ed. Cold Spring Harbor Press (2001)), or DNA chips, but are not limited thereto.

Meanwhile, the present invention provides a method for screening a cancer metastasis inhibiting agent, the method comprising:

(A) bringing a lysyl tRNA synthetase (KRS) or a KRS fragment comprising the C-terminal region thereof, syntenin, and a test agent into contact with one another;

(B) measuring the change in the binding level of the KRS or fragment thereof and syntenin; and

(C) bringing the test agent, which is determined to change the binding level of the KRS or fragment thereof and syntenin, into contact with cancer cells, to evaluate whether microvesicles containing the lysyl tRNA synthetase fragment of SEQ ID NO: 1 are secreted from the cancer cells.

The present inventors established the fact that the microvesicles (especially, KRS exosomes) of the present invention are secreted from cancer cells to create the cancer metastasis environment in tissues around the cancer cells, and that syntenin plays an important role in the secretion of the microvesicles. Specifically, the present inventors established the fact that the KRS increases its binding with syntenin-1 through the truncation of its N-terminus inside cells, and that the increased binding between syntenin and KRS is important in the secretion of KRS exosomes. The secreted KRS exosomes act on the recruitment of macrophage and neutrophils and increases the secretion of cytokines promoting cancer metastasis, resulting in playing an important role in cancer metastasis. Therefore, the present invention provides a method for screening a cancer metastasis inhibiting agent, the method comprising steps (A) to (C), on the basis of the foregoing secretion characteristics of the KRS fragment and the microvesicles containing the same, and the method will be described by each step.

In step (A), (i) a lysyl tRNA synthetase (KRS) or a KRS fragment comprising the C-terminal region thereof, (ii) syntenin, and (iii) a test agent are brought into contact with one another.

The specific amino acid sequence of KRS is not particularly limited as long as it is known as a lysyl tRNA synthetase in the art. For example, SEQ ID NO: 2 (Genbank Accession No. NP_005539.1), SEQ ID NO: 3 (Genbank Accession No. NP_001123561.1), and the like are known in the art. As used herein, the KRS in step (A) encompasses functional equivalents thereof.

The KRS fragment used in step (A) is characterized as a fragment comprising the C-terminal region of KRS, among pieces (fragments) of the full-length sequence of the KRS polypeptide. The C-terminal region of KRS may be composed of the amino acid sequence (VGTSV) represented by, preferably, SEQ ID NO: 6, but is not limited thereto.

Preferably, the KRS fragment (including the C-terminus region of KRS) that may be used in step (A) may be composed of the amino acid sequence of SEQ ID NO: 1, but is not limited thereto. In addition, as used herein, the KRS fragment in step (A) encompasses functional equivalents thereof.

The syntenin may be a syntenin-1 polypeptide comprising the amino acid sequence of, preferably, SEQ ID NO: 4, but is not limited thereto.

As used herein, the term “agent” or “test agent” encompasses any substance, molecule, element, compound, entity, or a combination thereof. For example, the term encompasses, but is not limited to, a protein, a polypeptide, a small organic molecule, a polysaccharide, a polynucleotide, and the like. Moreover, the term may include a natural product, a synthetic compound, a chemical compound, or a combination of two or more materials. Unless otherwise specified, the terms “agent”, “material”, and “compound” can be used interchangeably.

More specifically, the test agent that can be screened by the screening method of the present invention includes polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, saccharides, fatty acids, purine, pyrimidine or derivatives thereof, structural analogs, or combinations thereof. A certain test agent may be a synthetic material, while another test agent may be a natural material. The test agent may be obtained from a wide variety of sources including synthetic or natural compound libraries. Combinatorial libraries may be produced from several kinds of compounds that can be synthesized in a step-by-step manner. Compounds of multiple combinatorial libraries may be constructed by the encoded synthetic libraries (ESL) method (WO 95/12608, WO 93/06121, WO 94/08051, WO 95/395503, and WO 95/30642). Peptide libraries may be constructed by a phage display method (WO 1991/18980). Libraries of natural compounds in the form of bacteria, mold, plant, and animal extracts may be obtained from commercial sources or collected from fields. The known pharmacological agents may be applied to directed or random chemical modifications, such as acylation, alkylation, esterification, and amidification, in order to prepare structural analogs.

The test agent may be a naturally occurring protein or a fragment thereof. This test agent may be obtained from a natural source, for example, a cell or tissue lysate. The library of polypeptide agents may also be obtained, for example, from cDNA libraries, which are constructed by routine or commercially available methods. The test agent may be a peptide, such as a peptide having about 5-30 amino acids, preferably about 5-20 amino acids, and more preferably about 7-15 amino acids. The peptide may be a cleaved product of a naturally occurring protein, a random peptide, or a “biased” random peptide.

Alternatively, the test agent may be a “nucleic acid”. The nucleic acid test agent may be a naturally occurring nucleic acid, a random nucleic acid, or a “biased” random nucleic acid. For example, the cleaved product of a prokaryotic or eukaryotic genome may be used similar to the disclosure above.

In addition, the test agent may be a small molecule (e.g., a molecule with a molecular weight of about 1,000 or less). The high throughput assay may preferably be applied for screening a small-molecule modulating agent. A number of assays are available for such screening (Shultz, Bioorg. Med. Chem. Lett., 8:2409-2414, 1998; Weller, Mol. Drivers., 3:61-70, 1997; Fernandes, Curr. Opin. Chem. Biol., 2:597-603, 1998; and Sittampalam, Curr. Opin. Chem. Biol., 1:384-91, 1997).

Libraries of test agents to be screened by the method of the present invention may be constructed on the basis of structural studies of syntenin and KRS or a fragment or analog thereof. Such structural studies allow the identification of test agents that are more likely to bind to syntenin or KRS (or KRS fragments thereof). The three-dimensional structures of syntenin or KRS (or KRS fragments thereof) may be studied in a number of ways, e.g., crystal structure and molecular modeling. Methods of studying protein structures using x-ray crystallography are well known in the literature: Physical Bio-Chemistry, Van Holde, K. E. (Prentice-Hall, New Jersey 1971), pp. 221-239, and Physical Chemistry with Applications to the Life Sciences, D. Eisengerg & D. Crothers (Benjamin Cummings, Menlo Park 1979). Computer modeling of structures of syntenin or KRS provides another means for designing test agents to be screened. Molecular modeling methods are disclosed in literatures: U.S. Pat. No. 612,894 and U.S. Pat. No. 5,583,973. Further, the protein structures may also be determined using neutron diffraction and nuclear magnetic resonance (NMR). Physical Chemistry, 4th Ed. Moore, W. J. (Prentice-Hall, New Jersey 1972) and NMR of Proteins and Nucleic Acids, K. Wuthrich (Wiley-Interscience, New York 1986).

As used herein, the term “contact or contacting” has a general meaning, and refers to combining two or more agents (e.g., two polypeptides) or combining an agent and a cell (e.g., protein and cell). The contact may occur in vitro. For example, two or more agents are combined with each other or a test agent and cells or a test agent and a cell lysate are combined with each other in a test tube or a container. In addition, the contact may occur in cells or in situ. For example, recombinant polynucleotides encoding two polypeptides are co-expressed in a cell, so that two polypeptides are brought into contact with each other in a cell or a cell lysate.

In step (B), the change in the binding of the KRS or fragment thereof and syntenin, which are brought into contact with the test agent in step (A), is measured, while, from measurement results, a test agent showing a changed binding level of the KRS or fragment thereof and syntenin is selected.

The term “binding” may be the direct or indirect binding of the entire KRS or the fragment thereof having the amino acid sequence of SEQ ID NO: 1 and the syntenin protein. The indirect binding means that the binding between two proteins forms a complex via another mediating factor or together with the factor.

Herein, the change in the binding level between the KRS or fragment thereof and syntenin may be preferably a reduction in the binding level.

The reduction in the binding level means a removal, prevention, or suppression of the binding between the KRS or fragment thereof and syntenin. Specifically, the reduction in the binding level may be attained by: allowing the test agent to remove, prevent the generation of, or suppress the generation of the KRS or fragment thereof and syntenin to change the expression levels of the KRS or fragment thereof and syntenin; allowing the test agent to competitively or non-competitively binds to the KRS or fragment thereof and syntenin to change the interaction (binding) level therebetween; and allowing the test agent to act on a KRS (or fragment thereof)-syntenin protein composite, which has been already generated in cells, to remove the interaction (binding) between the KRS or fragment thereof and the syntenin in the composite. The competitive binding means that the test agent binds to an interaction (binding) site of the KRS or fragment thereof and the syntenin to remove, prevent, or suppress the interaction of the KRS or fragment thereof and syntenin, while the syntenin is characterized by interacting with the C-terminal region of the KRS or fragment thereof. The non-competitive binding means that the test agent binds to a region except for the interaction (binding) site of the KRS or fragment thereof and the syntenin to remove, prevent, or suppress the interaction of the KRS or fragment thereof and syntenin. That is, the present invention is directed to the screening of a test agent that inhibits the expression and inherent functions of the KRS or fragment thereof and syntenin, while at the same time (or independently) suppresses or reduces the intracellular interaction (binding) level of the KRS or fragment thereof and syntenin.

Preferably, the reduction in the binding level in the present invention may be preferably attained by a method wherein the test agent competitively or non-competitively binds to the KRS or fragment thereof and syntenin to change the interaction (binding) level therebetween, or a method wherein the test agent acts on a KRS (or fragment thereof)-syntenin protein composite, which has been already generated inside cells, to remove the interaction (binding) between the KRS or fragment thereof and the syntenin in the composite.

The test agents need not necessarily inhibit the expression and inherent functions of the KRS or fragment thereof and syntenin functionally, while merely the inhibition of the interaction (binding) between the KRS or fragment thereof and syntenin is enough.

The screening method according to the present invention may be performed by various methods known in the art, such as protein-protein binding assay in a labeled test tube (in vitro full-down assay), EMSA, immunoassay for protein binding, functional assay (phosphorylation assay, etc.), yeast 2-hybrid assay, non-immunoprecipitation assay, immunoprecipitation western blot assay, immuno-co-localization assay, and the like, but are not limited thereto.

For example, the yeast 2-hybrid assay may be carried out by using yeast expressing the KRS or fragment thereof and syntenin, or portions or homologues of these proteins, fused with the DNA-binding domain of bacteria repressor LexA or yeast GAL4 and the transactivation domain of the yeast GAL4 protein, respectively (KIM, M. J. et al., Nat. Gent., 34:330-336, 2003). The interaction between the KRS or fragment thereof and syntenin reconstructs a transactivator that induces the expression of a reporter gene under the control by a promoter having a regulatory sequence binding to the DNA-binding domain of LexA or GAL4 protein.

As described above, examples of the reporter gene may include genes that are known in the art and encode any detectable polypeptide (e.g., chloramphenicol acetyltransferase (CAT), luciferase, β-galactosidase, β-glucosidase, alkaline phosphatase, and green fluorescent protein (GFP)). If the binding between the KRS or fragment thereof and syntenin, or portions or homologues of these proteins is inhibited or weakened by the test agent, the reporter gene is not expressed, or is expressed less than under a normal condition.

Further, as the reporter gene, one that encodes a protein enabling the growth of yeast (i.e., the growth of yeast is inhibited if the reporter gene is not expressed) may be selected. For example, auxotropic genes that encode enzymes involved in biosynthesis for obtaining amino acids or nitrogen bases (e.g., yeast genes, such as ADE3 and HIS3, or equivalent genes derived from other species) may be used. In cases where the binding of the KRS or fragment thereof and syntenin, or portions or homologues of these proteins, which are expressed in this system, is inhibited by the test agent, the reporter gene is not expressed. Therefore, the growth of yeast is stopped or retarded under such a condition. The effect by the expression of the reporter gene may be observed with the naked eye or by using a device (e.g., a microscope).

In step (C), the agent selected in step (B) (i.e., the test agent showing a changed binding level of the KRS or fragment thereof and syntenin) is brought into contact with cancer cells to evaluate whether microvesicles containing the KRS fragment of SEQ ID NO: 1 are secreted from the cancer cells, and a test agent showing a reduced (inhibited) secretion of the microvesicles is selected.

In step (C), in order to investigate whether the microvesicles containing the KRS fragment of SEQ ID NO: 1 are secreted, materials (reagents) for detecting the presence of the KRS fragment represented by SEQ ID NO: 1 or microvesicles containing the same, and the amount thereof, the pattern thereof, or both may be used. The reagents are described as above, and methods for detecting a corresponding target material using the reagents are well known in the art.

Specifically, step (C) may be carried out by treating cancer cells with the test agent selected in step (B), culturing the cancer cells for a predetermined period of time, collecting the culture medium upon the completion of cell incubation, isolating the microvesicles from the culture medium, and measuring the level of the KRS fragment of SEQ ID NO: 1 in the microvesicles. The method wherein the purposed microvesicles are isolated from the sample and the level of the KRS fragment of SEQ ID NO: 1 is detected from the microvesicles may be referred to the above description.

In the method for screening a cancer metastasis inhibiting agent according to another aspect of the present invention, step (D) below may be further performed after steps (A) to (C):

(D) administering the test agent, which is determined to inhibit the secretion of the microvesicles containing the KRS fragment of SEQ ID NO; 1, to an animal with cancer, to evaluate whether the test agent exhibits an effect of preventing or treating cancer metastasis (that is, an effect of inhibiting cancer metastasis).

In step (D), the animal refers to a non-human animal, and may preferably be a non-human mammal.

### Advantageous Effects

The lysyl tRNA synthetase (KRS) fragment and the microvesicles containing the KRS fragment, provided in the present invention, are specific markers secreted extracellularly by cancer cells, and provide information necessary for the diagnosis of cancer, thereby easily performing the diagnosis of cancer using the same. Furthermore, the present invention provides a method for screening a cancer metastasis inhibiting agent on the basis of the secretion characteristics of the KRS fragment and the microvesicles containing the same from cancer cells, and the present invention can be favorably used in the development of materials that specifically inhibit the metastasis of cancer.

### BRIEF DESCRIPTION OF THE DRAWINGS

FIG. 1a illustrates western blot results verifying that N-terminus was truncated when KRS was secreted extracellularly. Myc-KRS or KRS-myc to be expressed was transfected into HCT116 cells, and after 24 hr, the transfectants were incubated with serum starvation media with or without TNF-α (10 ng/ml) for 12 hr. Secreted proteins were precipitated with TCA, and monitored by western blot using anti-myc antibody (WCL: whole cell lysate).

FIG. 1b illustrates results verifying that N-terminus was truncated when KRS was secreted extracellularly using strep-KRS-myc plasmid. The strep-KRS-myc plasmid was transfected into HCT116 cells, and after 24 hr, the transfectants were incubated in serum starvation media with or without TNF-α (10 ng/ml) for 12 hr. Secreted proteins were precipitated with TCA, and monitored by western blot using anti-STREP antibody and anti-myc antibody (WCL: whole cell lysate).

FIG. 1c illustrates results verifying the cleavage between 12-13 a.a. in KRS protein. Myc-KRS wild type and myc-▴N12 (13-597 a.a. mutant) were transfected into HCT116 cells, and after 24 hr, the transfectants were incubated with serum starvation media with or without TNF-α (10 ng/ml) for 12 hr. Secreted proteins were precipitated with TCA, and monitored by western blot using anti-myc antibody (WCL: whole cell lysate).

FIG. 1d illustrates results investigating the amount of KRS truncation for a pre-determined time in serum starvation condition or serum starvation+TNF-alpha condition, respectively. GFP-KRS was overexpressed in HCT116 cells, and after 24 hr, the cells were incubated with serum starvation media with or without TNF-α (10 ng/ml) for 0, 30, and 60 min. Thereafter, GFP-KRS and GFP proteins (truncated GFP, N-terminus of KRS being truncated in GFP-KRS conjugate) were detected by western blot using anti-GFP antibody in whole cell lysates.

FIG. 1e illustrates luciferase assay results investigating the KRS truncation according to serum starvation treatment time. Specifically, it was investigated whether KRS was truncated by the starvation condition by using N-renilla-KRS-C-renilla vector. N-renilla-KRS-C-renilla vector and firefly luciferase vector were transfected into HCT116 cells, and after 24 hr, the transfectants were incubated in serum starvation media according to the time (0 hr, 3 hr, and 6 hr). Then, the renilla/firefly luciferase activity in each sample was determined.

FIG. 2a illustrates the results of KRS multiple-alignment using BioEdit. Caspase-cleavage consensus and eukaryote-specific expansion domains were indicated in this figure.

FIG. 2b illustrates western blot results investigating the secretion of KRS by Pan-caspase inhibiting agent. The Pan-caspase inhibiting agent, Z-VAD-FMK (14 uM), was added to starved HCT116 cells. After incubation for 12 hr, KRS secretion was monitored by western blot using anti-KRS antibody (WCL: whole cell lysate).

FIG. 2c illustrates western blot results investigating the cleavage of KRS by Pan-caspase inhibiting agent. The results show that the intracellular cleavage of KRS was inhibited by Pan-caspase inhibiting agent. GFP-KRS overexpressed cells were incubated in serum starvation media with the pan-caspase inhibiting agent (Z-VAD-FMK, 14 uM) for 1 hr. The cleavage of KRS was detected by western blot using anti-GFP antibody.

FIG. 2d illustrates the western blot results investigating the secretion of KRS through partial mutation (D12A) of KRS sequence recognized by caspase. That is, KRS-myc wild type (WT) or D12A mutant (variant in which Asp, i.e., 12th amino acid of KRS WT, is replaced with Ala) was used to test whether the secretion of KRS is caspase-dependent. KRS WT with myc-tagged C-terminus or D12A mutant to be expressed was transfected into HCT 116 cells, and after 24 hr, the transfectants were incubated in serum starvation media for 12 hr, and the secretion of KRS was monitored by western blot using anti-myc antibody (WCL: whole cell lysate).

FIG. 2e illustrates western blot results investigating the cleavage of KRS through partial mutation (D12A) of KRS sequence recognized by caspase. GFP-KRS WT or D12A mutant thereof was transfected and overexpressed in HCT116 cells, and after 24 hr, the transfectants were incubated in serum starvation media for 1 hr. GFP-KRS and cleaved GFP were detected by western blot using anti-GFP antibody.

FIG. 2f illustrates western blot results investigating the secretion of KRS by the treatment with caspase-3, -6, -8, and -9 inhibiting agents. HCT116 cells were incubated in serum starvation media treated with respective caspase-3, -6, -8, and -9 inhibiting agents (Z-VAD-DQMD (3), Z-VAD-VEID (6), Z-VAD-IETD (8), and Z-VAD-LEHD (9)) for 12 hr, and then, the extracellular secretion of KRS was monitored by western blot using anti-KRS antibody (WCL: whole cell lysate).

FIG. 2g illustrates western blot results investigating the secretion of KRS by the treatment with caspase-3, -6, -8, -9 siRNA. HCT116 cells were transfected with caspase-3, -6, -8, -9 specific siRNAs and non-specific siRNA control, and after 48 hr, the transfectants were incubated in serum starvation media, and then the secretion of KRS was monitored by western blot using anti-KRS antibody (WCL: whole cell lysate).

FIG. 2h illustrates western blot results investigating the expression levels of Caspase-3, -6, -8, -9 in serum starvation condition. HCT116 cells were incubated in serum starvation media for a pre-determined time, and then the expression level of each caspase was monitored in protein lysate.

FIG. 2i illustrates results verifying that the increased caspase-8 leads to an increase in the N-terminus truncation of KRS, by investigating the amount of KRS cleaved in caspase-8 overexpressed condition using western blot. Caspase-8 and GFP-KRS were overexpressed in HCT116 cells, and after 24 hr, the cells were incubated in serum starvation media for 1 hr, and then the cleavage of KRS in intracellular space was monitored by western blot using anti-GFP antibody.

FIG. 3a illustrates the results of multiple alignment for PDZ binding motif at C-terminus of KRS.

FIG. 3b illustrates immunoprecipitation and western blot results of the binding of KRS and syntenin-1 in starvation condition and/or TNF-alpha treatment. The results verified that the interaction between KRS and syntenin-1 was induced by starvation. HCT116 cells were incubated in serum starvation media condition or in TNF-alpha containing serum starvation media for 1 hr. The interaction between KRS and syntenin-1 was monitored by immunoprecipitation (IP) of syntenin-1 and western blot using anti-KRS antibody.

FIG. 3c illustrates western blot results investigating the binding between KRS and syntenin-1 upon the treatment with caspase-8 inhibiting agent. It was verified that the KRS-syntenin-1 interaction was reduced by caspase-8 inhibiting agent treatment. Cells were incubated in serum starvation medium treated with or without caspase-8 inhibiting agent (Z-VAD-IETD), and then the interaction between KRS and syntenin-1 was monitored by immunoprecipitation (IP) of syntenin-1 and western blot using anti-KRS antibody.

FIG. 3d illustrates results investigating the interaction between KRS and syntenin-1 using D12A mutant. C-terminus myc tagged KRS WT and D12A mutant were transfected and overexpressed in cells, and after 24 hr, the transfectants were incubated in serum starvation media for 1 hr, followed by precipitation using anti-myc antibody, and then KRS-bound syntenin-1 was monitored by western blot (mock: control with empty vector introduced into cells).

FIG. 3e illustrates results investigating the interaction (binding) of KRS and syntenin-1 using bimolecular fluorescence complementation (BiFC) assay. KRS WT-VN173 or D12A mutant-VN173 and syntenin-1-VC15 were transfected and overexpressed in cells, and after 24 hr, the transfectants were incubated in serum starvation media for 4 hr, and then monitored using fluorescence microscope. Here, one of test groups was treated with caspase-8 inhibiting agent. Flag-KRS was detected by anti-flag-antibody.

FIG. 3f illustrates western blot results investigating the secretion of KRS by syntenin-1-specific siRNA. It was verified that the secretion of KRS was dependent on syntenin-1. Syntenin-1 was down regulated by syntenin-1 specific siRNA in HCT116 cells. After 48 hr, the cells were incubated in serum starvation media for 12 hr. The secreted KRS was precipitated with TCA, and detected by western blot using anti-KRS antibody (con: siRNA control treatment, syn: syntenin-1 specific siRNA treatment, WCL: whole cell lysate).

FIG. 3g illustrates western blot results investigating the binding of syntenin-1 and the deletion mutant (▴c5(1-592 a.a)) in which the C-terminus of KRS was truncated. KRS WT and C-terminus deletion mutant (▴c5(1-592 a.a)) were used to investigate the interaction with syntenin-1 and the KRS secretion. HCT116 cells were transfected with KRS WT-myc or deletion mutant (▴c5(1-592 a.a))-myc. After 24 hr, the cell lysate was immunoprecipitated (IP) using anti-syntenin-1 antibody, and then the syntenin-1 bound KRS was monitored in the precipitant by western blot using anti-myc-antibody (WCL: whole cell lysate).

FIG. 3h illustrates western blot results investigating the effect of KRS deletion mutant (▴c5(1-592 a.a)) on KRS secretion. HCT116 cells were transfected with KRS WT and deletion mutant (▴c5(1-592 a.a)), and after 24 hr, the transfectants were incubated in serum starvation media for 12 hr. Proteins secreted in culture media were precipitated with TCA, and then the amount of secreted KRS was detected by western blot using anti-myc antibody.

FIG. 4a shows electron microscopy images of microvesicles isolated from KRS-secreted media. HCT116 cells were incubated in serum starvation media for 12 hr, and extracellularly secreted microvesicles were isolated by centrifugation at 100,000 g, and images thereof were checked using electron microscopy.

FIG. 4b illustrates the mean size of microvesicles isolated from KRS-secreted media. HCT116 cells were incubated in serum starvation media for 12 hr, and microvesicles were isolated from the culture media by centrifugation at 100,000 g. The size of the isolated microvesicles was measured using dynamic light scattering.

FIG. 4c illustrates results verifying the density of KRS-detected microvesicles using opti-prep gradient assay. HCT116 cells were incubated in serum starvation media for 12 hr, and the microvesicles isolated from the culture media were loaded on the opti-prep gradient to obtain a total of nine fractions, which were then analyzed by western blot using anti-KRS antibody and anti-syntenin-1 antibody.

FIG. 4d illustrates results verifying that the KRS exosome secretion was dependent on syntenin-1, by monitoring the KRS exosome secretion using western blot after si-syntenin treatment. Syntenin-1 was down regulated by syntenin-1 specific siRNA in HCT116 cells. After 48 hr, the cells were incubated in serum starvation media for 12 hr. Secreted exosomes were purified by centrifugation at 100,000 g, and proteins were monitored by western blot (si-Con: siRNA control treatment having no effect on gene expression, si-syn: syntenin-1 specific siRNA treatment, WCL: whole cell lysate).

FIG. 4e illustrates western blot results investigating the effect of KRS deletion mutant (▴c5(1-592 a.a)) on KRS exosome secretion. KRS WT-myc or deletion mutant (▴c5(1-592 a.a))-myc was used to investigate the interaction with syntenin-1 and the KRS exosome secretion. HCT116 cells were transfected with KRS WT-myc or deletion mutant (▴c5(1-592 a.a))-myc. After 24 hr, the transfectants were incubated in serum starvation media for 12 hr. The purified exosomes were analyzed by western blot (WCL: whole cell lysate).

FIG. 4f illustrates western blot results investigating the effect of D12A mutation on KRS exosome secretion. In order to investigate the interaction between KRS truncation and KRS exosome secretion, D12A mutant was used. C-terminus myc tagging KRS WT or D12A mutant thereof was transfected and overexpressed in HCT116 cells. After 24 hr, the transfectants were incubated in serum starvation media for 12 hr, and exosomes were isolated by centrifugation at 100,000 g, and the proteins thereof were monitored by western blot (WCL: whole cell lysate).

FIG. 5a illustrates TNF-alpha ELISA results investigating TNF-alpha secretion by treatment of macrophages with KRS WT, truncated KRS (▴N12(13-597 a.a)), or KRS exosomes. RAW 264.7 cells were incubated together with 100 nM KRS WT, truncated KRS (▴N12(13-597 a.a)) protein, and KRS exosomes (0.05, 0.5, 5 ug), and the secreted TNF-alpha was analyzed.

FIG. 5b illustrates results investigating cell migration by treatment of macrophages with KRS WT, truncated KRS (▴N12(13-597 a.a)), or KRS exosomes. The cell migration by KRS exosome treatment was monitored by wound-healing assay. The RAW 264.7 cell monolayer was once scratched, and then treated with 100 nM KRS proteins (WT, ▴N12 each) or KRS exosomes (0.05, 0.5, 5 ug) in their respective concentrations. After 12 hr, the cell migration was observed by a microscope.

FIG. 6a illustrates immunoblotting results of samples obtained by purifying exosomes from si-con or si-KRS treated HCT116 cells.

FIG. 6b illustrates analysis results of TNF-alpha secretion by TNF-alpha ELISA, when RAW 264.7 cells were incubated together with 100 nM ▴N12 KRS protein or exosomes (5 ug/ml) purified from si-con or si-KRS treated HCT116 cells.

FIG. 6c illustrates analysis results of cell migration effect by transwell migration assay, when RAW 264.7 cells were incubated together with 100 nM ▴N12 KRS protein or exosomes (5 ug/ml) purified from si-con or si-KRS treated HCT116 cells (microscopic observation images (left) and quantified percentage of migrated cells (right)).

FIG. 6d shows intravital images (left) obtained by using KRS WT-myc or D12A mutant-myc overexpressed B16F10 cells and quantified results (right) of green fluorescent intensities on the images. After the B16F10 cells were injected into mouse ears, and then images according to the time were obtained at 0, 30, 60, 90 min (red: cells, green: macrophages and neutrophils).

FIG. 6e illustrates evaluation results of levels of respective cytokines secreted from macrophages treated with 100 nM KRS proteins (WT, ▴N12 each) or KRS exosomes (5 ug/ml), using luminex screening assays (bead-based multiplex kits) (Cont: non-treatment control, Exo: KRS exosome treatment group).

### MODE FOR CARRYING OUT THE INVENTION

Hereinafter, the present invention will be described in detail.

However, the following examples are merely for illustrating the present invention and are not intended to limit the scope of the present invention.

<Methods>

1. Cell Incubation and Materials

HCT116 cells were incubated in 5% CO₂ incubator at 37° C. using RPMI media (together with 25 mM HEPES and L-glutamine, Hyclone) supplemented with 10% fetal bovine serum (FBS) and 50 μg/mL penicillin and streptomycin. RAW264.7 cells were incubated in 5% CO₂ incubator at 37° C. using high glucose DMEM (together with 2.5 g of porecine trypsin, 4.00 mM L-glutamate, 400 mg/L glutamine, and sodium pyruvate, Hyclone) supplemented with 10% fetal bovine serum (FBS) and 50 pg/mL penicillin and streptomycin. Human TNF-alpha (Sigma, USA) treatment was conducted at a concentration of 10 ng/ml in serum-free condition. si-RNA against syntenin-1 was obtained from Santa Cruz (sc-42164). si-RNA against KRS was obtained from Invitrogen; KRS si-RNA sequence (Cat. No/Lot No. 10620318-277773 C07, C08: KARS shss105656: GGGAAGACCCAUACCCACACAAGUU, AACUUGUGUGGGUAUGGGUCUUCCC). si-RNAs specific to caspase-3, -6, -8, -9 were obtained from Sigma-aldrich. Stealth universal RNAi (Santa Cruz) was used as a non-specific control, and Lipofectamine™ 2000 Transfection reagent (Invitrogen, Cat. No. 18324-012) was used for transfection according to the manufacturer's protocol. Here, caspase-3 inhibitor (Cat No. 219002), caspase-6 inhibitor (Cat No. 218757), caspase-8 inhibitor (Cat No. 368055), and caspase-9 inhibitor (Cat No. 218776) were obtained from Calbiochem. In addition, the caspase inhibiting agent treatment was conducted at a concentration of 14 uM under serum-free condition.

2. Western Blot and Immunoprecipitation

The cells were lysed with 50 mM Tris-HCl (pH 7.4) buffer containing 150 mM NaCl, 10 mM NaF, 12 mM beta-glycerophosphate, 1 mM EDTA, 1% NP-40, 10% glycerol, and protease inhibiting agent. Then, the supernatant was dissolved in SDS sample buffer, followed by separation using SDS-PAGE. For immunoblotting of endogenous KRS, anti-KRS antibody was used. Antibodies against Hsp90, syntenin-1, GFP, myc, caspase-3, -6, -8, -9, while syntenin-1 were purchased from Santa Cruz, and antibodies against alix were purchased from Cell Signaling. The anti-KRS antibody was manufactured by ordinary procedures in which KRS protein (Genbank Accession No. NP_005539.1) represented by SEQ ID NO: 2 was injected into New Zealand white rabbits to induce immune response and their antibodies were obtained.

For immunoprecipitation, the cells were lysed at 4° C. in 50 mM HEPES (pH 7.4) buffer containing 150 mM NaCl, 0.5% NP-40, 2 mM EDTA, 5% glycerol and protease inhibiting agent (Calbiochem, San Diego, Calif., USA). Protein extracts were cultured together with protein-specific antibodies thereagainst with stirring at 4° C. In addition, protein G agarose was added. 4 hr after the addition of the protein G agarose, precipitate samples were obtained by centrifugation. The precipitate samples were washed three times for 5 min using cool lysis buffer. The precipitates were separated by SDS-PAGE.

3. KRS Secretion Test

HCT116 cells were incubated in RPMI media containing 10% FBS (Hyclone), and were grown to 60% confluency on 60-mm dish. The cells were washed twice with PBS, incubated in serum-free RPMI media, and treated with 10 ng/ml TNF-α for 12 hr. The supernatant of the cell culture liquid was cautiously collected, followed by centrifugation at 500 g for 10 min. The supernatant thus obtained was again centrifuged at 10,000 g for 30 min, thereby removing membrane organelles. Then, 12% TCA was added to the supernatant, followed by culture at 4° C. for 12 hr, so the supernatant was subjected to precipitation treatment. After the precipitation treatment, the supernatant was centrifuged at 18,000 g for 15 min, and the upper part was then discarded and the remaining pellets were neutralized with 100 mM HEPES (pH 8.0), and then 5× sample buffer was added to perform separation using SDS-PAGE. The products separated by SDS-PAGE were western blotted using anti-KRS antibody.

4. Preparation of Human Full Length KRS and Truncated KRS Protein (▴N12 KRS)

First, cDNA encoding Human KRS of SEQ ID NO: 2 was subcloned into pET-28a (Novagen) using restriction enzymes, EcoRI and XhoI, and then was introduced and overexpressed into Escherichia coli BL21 (DE3). Then, his-tagged KRS was purified using nickel affinity (Invitrogen) and Mono Q ion-exchange chromatography according to the manufacturer's protocol. For the removal of lipopolysaccharide (LPS), KRS-containing solution was dialyzed with pyrogen-free buffer. For the removal of still remaining LPS, the KRS solution was again dialyzed with PBS containing 20% glycerol, and filtered through Posidyne membrane (Pall Gelman Laboratory).

5. Immunofluorescent Staining

KRS-VN173 plasmid and syntenin-1-VC155 plasmid were all transduced in HCT116 cells, and the cells were incubated in starvation condition or serum-free condition for 4 hr. The HCT116 cells were located on 9-mm coverslip, fixed using 4% paraformaldehyde, and washed shortly with cool PBS. The cells were incubated in 5% BSA blocking buffer for 1 hr, and DAPI stained for 10 min. The cells were washed six times with cool PBS five times for 5 min for each time, and then mounted on slide glass. Thereafter, the samples were observed using confocal laser scanning microscope A1 (Nikon).

6. Microvesicle Isolation

HCT116 cells were incubated for a predetermined period of time in each treatment condition (especially, serum-starvation condition), and then the media were separated and consecutively centrifuged. Centrifugation was conducted three times at 500 g (10 min), 10,000 g (30 min), and 100,000 g (90 min) to form microvesicle pellets. The amount of microvesicle proteins was determined by using Bradford assay.

7. Opti-Prep Gradient Centrifugation

In order to measure density of microvesicles, microvesicles pelletized at 100,000 g were loaded onto the continuous opti-prep gradient, and then centrifuged at 150,000 g for 15 hr. Nine fractions were obtained, followed by density measurement using a refractive index, and then resuspended using SDS-PAGE sample buffer, and subjected to immunoblotting using specific antibodies.

8. Electron Microscopic Observation

For negative staining, isolated microvesicles were diluted 5-fold with PBS. Following dilution, 5 μl was applied to a glow-discharged carbon-coated grid (Harrick Plasma, USA) for 3 min in air, and the grid was negatively stained using 1% uranyl acetate (see Jung, H. S., et. al., Mol. Biol. Cell: 19; 3234-3242, 2008). The same procedure was used for all samples. For immuno-electronic microscopy, the microvesicles were mixed with polyclonal anti-GRS antibody for 6 hr or less, and then were allowed to bind with secondary rabbit antibody conjugated with 6 nm gold particles (JIRE, U.K.). Thereafter, the mixture was left on ice for 12 hr, and then negatively stained as described above. The grids were tested using a Technai G2 Spirit Twin TEM (FEI, USA) operated at 120 kV. Images were recorded on 4K×4K Ultrascan 895 CCD (Gatan, USA).

9. Dynamic Light Scattering

The secreted microvesicles were obtained and resuspended in PBS. Thereafter, the particle size was measured by light scattering spectrophotometer ELS-Z (Otsuka Electronics, Japan). Measurement was performed in automatic mode after equilibration for 5 min at 20° C. Data were processed with manufacturer's software in multiple narrow modes.

10. BiFC-Renilla Luciferase Assay

The Renilla Luciferase Reporter Assay System (Promega, Madison, Wis.) was used to measure the luciferase activity. In addition, firefly luciferase vector was used as a control. The luciferase activity was calculated using FLUOstar OPTIMA (BMG LABTECH). After BiFC-renilla luciferase KRS plasmid and firefly luciferase plasmid were introduced, the cells were incubated in serum free media. After the media were removed, the cells were washed using PBS. 80 ul/well of lysis buffer (Promega, Madison, Wis.) was added to each well, and gently stirred at room temperature for 15 min. Cell lysates were harvested, and used for luciferase assay. First, 20 ul of the cell lysate was transferred on the 2 white opaque 96-well plates (Falcon, 353296). In addition, Firefly and Renilla luciferin were transferred on each of the 2 white opaque 96-well plates. After the injector dispensing assay reagent was injected into each well, 2-second pre-measurement delay and thereafter 10-second measurement period were given for each luminescence reading. Luciferase assays were based on the Renilla/Firefly ratio to normalize the number of cells and the transformation efficiency.

11. Wound Healing Assay

RAW264.7 cells were dispensed on the coverslip, and grown to >95% confluency. Subsequently, the RAW 264.7 monolayer was scratched to create wounds, and then the wounds were treated with 100 nM KRS proteins (WT, ▴N12) or KRS exosomes (0.05, 0.5, 5 ug) in their respective concentrations, followed by incubation for 12 hr. The cell morphology of cells was observed using microscopy.

12. TNF-Alpha Secretion ELISA Assay

RAW264.7 cells (2×10⁴) were incubated in 24-well plate containing DMEM supplemented with 10% FBS and 1% antibiotic for 12 hr, and starved in serum starvation media for 2 hr. 100 nM KRS protein (WT, ▴N12 each) and KRS exosomes (0.05, 0.5, 5 ug) were added, followed by treatment for 6 hr. Thereafter, cell media were collected by centrifugation at 3,000 g for 5 min. TNF-alpha secreted from the cells was detected using the TNF-alpha ELISA kit (Pharmingen, BD Science) according to the manufacturer's protocol.

13. Transwell Migration Assay

The transwell cell culture chamber 24-well plate (6.5 mm insert with 5.0 uM polycarbonate membrane) was purchased from Costar. The 5 uM inserts were coated with 10 uL of 0.5 mg/mL gelatin (Sigma), and dried under UV overnight. RAW264.7 cells were suspended in serum-free DMEM, and added to the inserts at 1×10⁵ cells. Each well was treated with exosomes (5 ug/ml) purified from cells treated with BSA (100 nM), KRS (▴N12) (100 nM), si-control, or si-KRS, and incubated in 5% CO₂ incubator at 37° C. for 8 hr. The inserts were washed twice using cool PBS, and the cells were fixed with a solution containing 70% methanol and 30% PBS for 30 min. Subsequently, the inserts were washed three times with PBS, and stained with hematoxylin (Sigma) for 30 min. The inserts were washed three times with distilled water, and non-migrated cells were removed using the cotton swab. The membrane was collected using a razor blade, and mounted on the microslide using Gel Mount (Biomeda). The images of migrated cells were obtained using Optinity microscope installed with Top view program.

14. Intravital Imaging

14.1 Imaging System and Imaging Procedure

In order to visualize that macrophage/neutrophil recruitment was increased by KRS, the custom-built laser-scanning confocal microscope identical to one used in previous study of K. Choe et al., 2013 was used. Three CW lasers for 488 nm (MLD488 60 mW, Cobolt), 561 nm (Jive™ 50 mW, Cobolt), and 640 nm (MLD640 100 mW, Cobolt) were used as excitation sources. For the implementation of 2D scanning, the fast-rotating polygonal mirror (MC-5, aluminum coated, Lincoln Laser) and galvanometer (6230H, Cambridge Technology) were used. For simultaneous detection of three-color fluorescent signals, the High-sensitive photomultiplier tube (R9110, Hamamatsu) was used. Three detection channels were divided by dichroic mirrors (FF01-442/46-25, FF02-525/50-25, FF01-585/40-25, FF01-685/40-25, Semrock) and bandpass filters (FF484-FDi01, FF560-Di01, FF649-Di01, Semrock). Electric signals obtained from PMT were digitalized by the 8-bit 3-channel frame grabber (Solios, Matrox). The Field of view (FOV) of images obtained from 20× (LUMFLN60XW, NA1.1, Olympus) was 500×500 μm². 512×512 pixel images were obtained from the imaging system, and then subjected to XY-shift compensation using Matlab (Mathworks). For accurate adjustment of sample position, the motorized XYZ translational stage (MPC-200-ROE, Sutter Instrument) with 1 μm resolution was used during the imaging procedure.

14.2 Animal Model

In the present study, LysM-GFP (Lysozyme M-GFP) mice endogenously exhibiting GFP fluorescence in macrophages and neutrophils were used (N. Faust et al., 2000). 12-20 week-old male LysM-GFP mice were anesthetized by intraperitoneal injection of Zoletil® (30 mg/kg) and xylazine (Rompun®, 10 mg/kg). The body temperature was maintained at 37° C. using the homeothermic controller (PhysioSuite™, RightTemp™, Kent Scientific) during the imaging procedure. In order to remove the possibility of occurrence of immune response due to depilation, the mouse ear skin was shaved at least 12 hr prior to imaging.

14.3 Intravital Imaging Using Cells

In order to investigate the effect of KRS secretion increased by tumor cells, B16F10 cells were transfected with KRS-myc, D12A-myc, or empty vector using Lipofectamine 3000 (Invitrogen, 11668027). The transgenic B16F10 cells were fluorescence-labeled with the Vybrant DiD solution (V-22887, Life Technologies), as a lipophilic fluorescent dye, and this procedure was performed by adding 5 μL DiD of the solution per 1 ml of cell media and incubating the cells. After washing three times with PBS, the labeled cells were suspended in PBS solution for preparation (0.4 million cells/μL). 4×10⁴ cells were injected into the mouse ear skin using the 31G microinjector. In order to visualize macrophage/neutrophil recruitment along the location of the cell injection, time-lapse images were taken by 90 min after the injection at intervals of 30 min.

15. Luminex Screening Assays (Bead-Based Multiplex Kits)

RAW264.7 cells were incubated in 12-well plate using DMEM media containing 10% FBS and 1% antibiotic for 12 hr, and starved in serum starvation media for 2 hr. KRS proteins (WT, ▴N12 each) and KRS exosomes (5 ug) in different amounts were respectively added to the media. After 12 hr, conditioned media were collected, and spun down through centrifugation at 3,000 g for 10 min. For multiplex assay, premixed beads for TNF-alpha, mCRG-2, IL-6, mIL-lbeta, mIL-12, mIL-10, MMP9, INF-gamma, mMIP3a, and CXCL10 were purchased from R&D Science, and used according to the manufacturer's protocol. Each sample were analyzed by BioRad Bioplex 200 system and software.

### Example 1

### Truncation of KRS N-Terminus in KRS Secretion

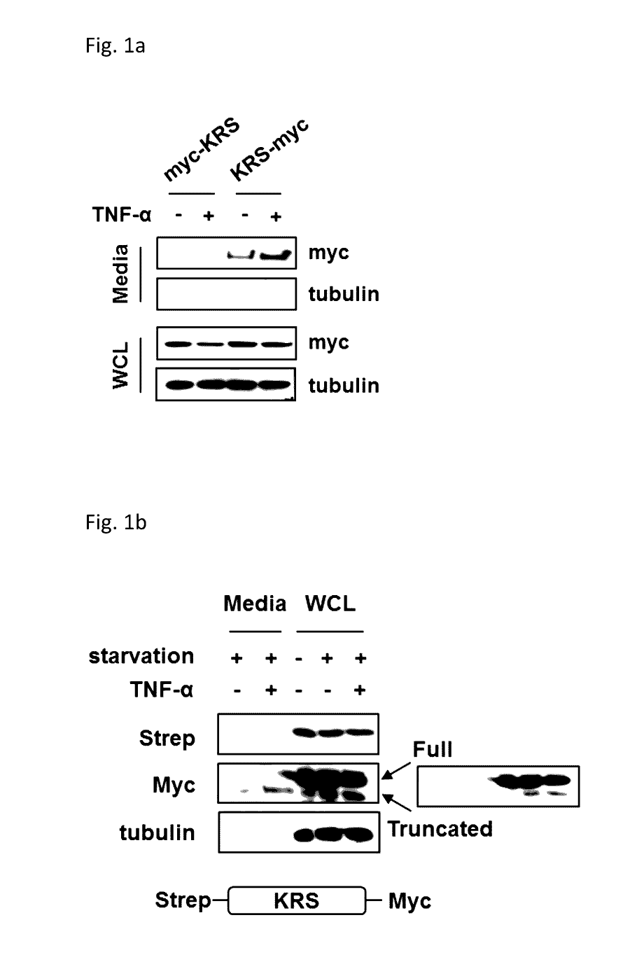

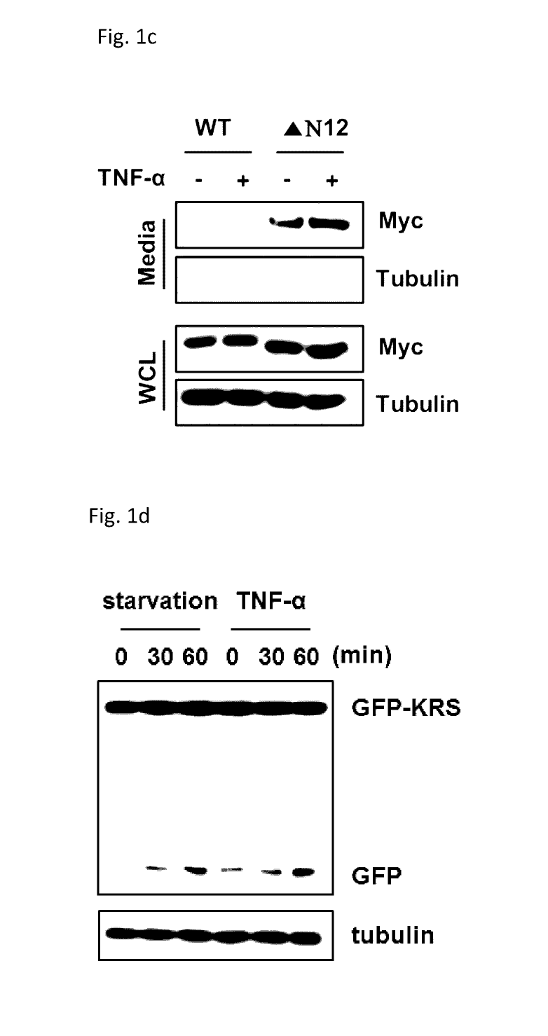

It has been known that KRS proteins are secreted from cancer cells to increase TNF-alpha secretion and macrophage migration through macrophage, causing inflammation responses. It has been known that KRS is secreted at the serum starvation, and the secretion of KRS is increased upon a simultaneous treatment of TNF-alpha. Little has been known about how the KRS is secreted. Herein, when Myc-KRS and KRS-myc plasmid were transfected into HCT116 cells and then KRS secretion was observed by western blot, the truncation of its N-terminus was confirmed at the time of KRS secretion (see FIG. 1a). In order to investigate these results, the test was performed after the plasmid composed of KRS with strep-tagged N-terminus and myc-tagged C-terminus was constructed. As a result, it was again verified that the N-terminus of KRS was truncated both when treated with serum starvation and when treated with serum starvation and TNF-alpha together (see FIG. 1b). Herein, in order to ensure the cleaved portion, a preliminary test was performed. As a result, the cleavage between 12th and 13th a.a. was confirmed. In order to verify these results, myc-KRS WT (1-597) or myc-KRS mutant (13-597, also designated by ▴N12 and meaning the peptide of SEQ ID NO: 1) was transfected into cells before the secretion test was performed. As a result, the cleavage between 12th and 13th a.a was again confirmed (see FIG. 1c). As stated above, KRS was secreted by starvation, and the KRS secretion was increased by starvation+TNF-alpha. As shown in FIG. 1b, it was verified that the amount of KRS truncation was constant for both cases of starvation and TNF-alpha treatment. In order to investigate those facts again and ensure the signal of KRS truncation, GFP-KRS was used. GFP-KRS was transfected into HCT116 cells, which were then treated with starvation and TNF-alpha. In starvation and TNF-alpha treatments for each time, the amount of KRS truncation was the same (see FIG. 1d). These results verified that starvation is a signal for truncating KRS. In order to ensure the KRS truncation at the time of starvation, N-renilla-KRS-C-renilla plasmid was used. This plasmid has renilla luciferase activity at ordinary times through the combination of one half of renilla at the KRS N-terminus and the other half of renilla at the KRS C-terminus, but has no activity in the absence of any one of the two. N-renilla-KRS-C-renilla plasmid and firefly luciferase plasmid were transfected into HCT116 cells before the test was performed. As a result, it was confirmed that the renilla luciferase activity was reduced according to the time of starvation, and the above results confirmed that the KRS N-terminus was truncated (see FIG. 1e). These results confirmed that the truncation procedure was necessary for the secretion of KRS.

### Example 2

### KRS Cleaved by Cascase-8

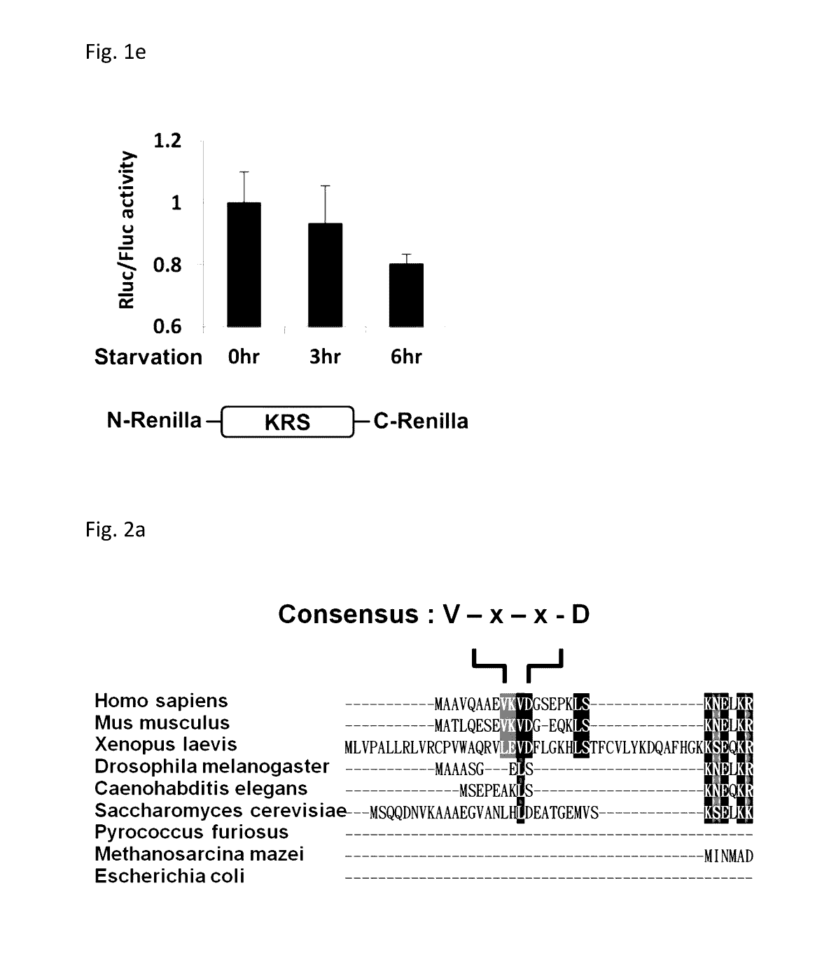

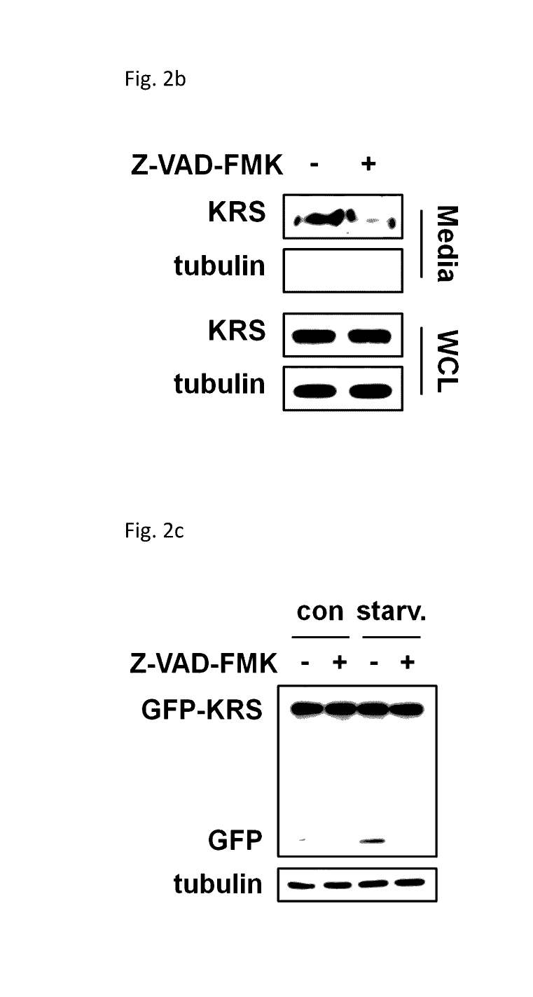

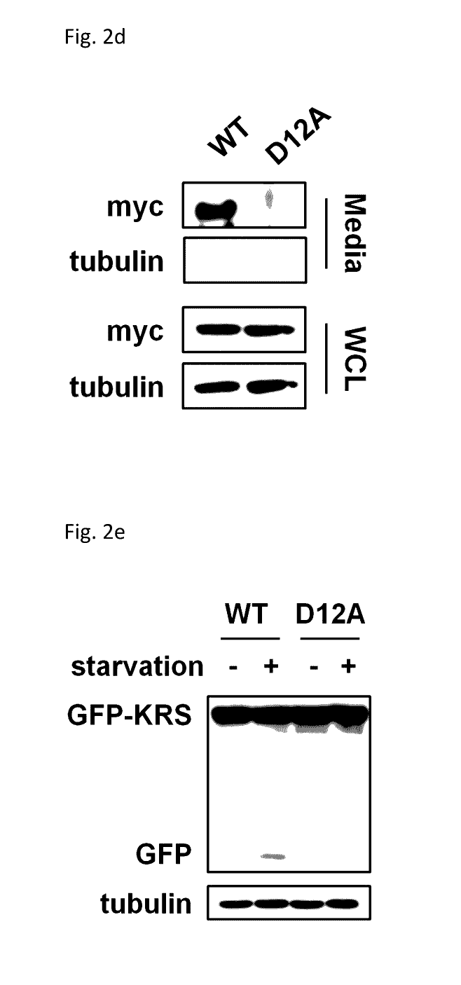

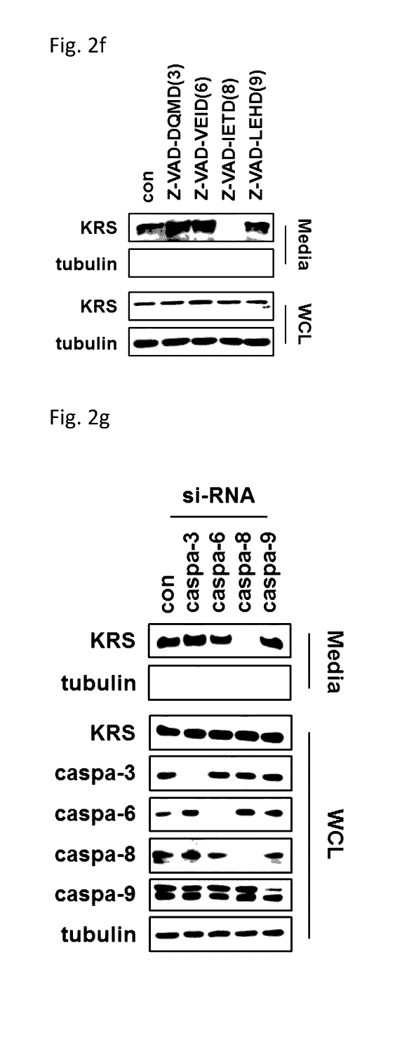

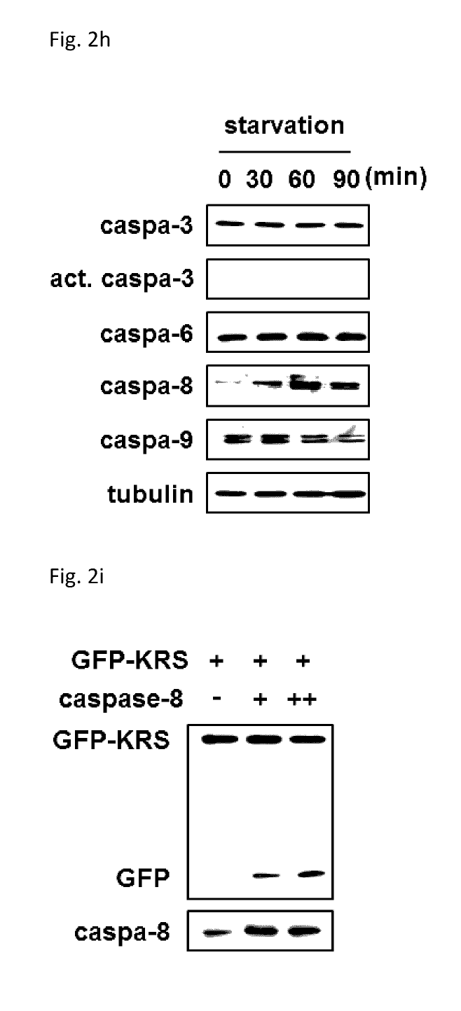

A large number of proteases exist in cells, and particular proteases recognize their recognizable particular sequences to perform a cleavage procedure. Herein, in order to find KRS-cleaving proteases, it was investigated whether there is any particular sequence in the KRS sequence. As a result of multiple alignment, caspase-cleavable sequences conserved in higher eukaryotes were found (see FIG. 2a). In order to investigate the effect of caspase on KRS, pan-caspase inhibitor was used. It was investigated whether KRS secretion and KRS cleavage were reduced after the treatment with pan-caspase inhibitor. As a result of pan-caspase inhibitor treatment, it was verified that the KRS secretion and KRS cleavage were reduced (see FIGS. 2b and 2c). The secretion of KRS unrecognizable by caspase and the reduction of the KRS cleavage were investigated through partial mutation (D12A) of the KRS sequence recognized by caspase. It could be seen from test results that the secretion and cleavage were reduced for D12A mutant (see FIGS. 2d and 2e). It could be seen through the above two tests that the caspase was involved in the cleavage of KRS and the cleavage by the caspase increased the secretion of KRS. Among various caspases, the sequences of KRS have a possibility of being cleaved by caspase-3, -6, and -8. Particularly, KRS secretion by the treatment with caspase-3, -6, -8, -inhibitors was investigated. It could be seen from test results that only caspase-8 inhibitor inhibited KRS secretion (see FIG. 2f). In addition, from the results of monitoring KRS secretion after the expression level of particular caspase protein was reduced using siRNA, it was verified that the secretion of KRS was reduced only when caspase-8 was reduced, which was identical to the results of tests using caspase inhibitor (see FIG. 2g). If caspase-8 functions to cleave KRS at the time of KRS secretion occurring in starvation environment, there would be a change in the expression level and activity in the starvation environment. It was verified that the amount of caspase-8 was increased over time unlike caspase-3, -6, -9 in the starvation condition inducing the secretion of KRS (see FIG. 2h). When the amount of KRS cleaved after caspase-8 overexpression was investigated using GFP-KRS, it was found that the amount of KRS cleaved was increased with the increasing amount of caspase-8 (see FIG. 2i). The above test results validated that caspase-8 was increased, leading to KRS cleavage in the starvation environment. Through the tests, it was verified that caspase-8 functions to cleave KRS and this cleavage is an important procedure necessary for KRS secretion. The above results validated that a front region of the 13th a.a of the N-terminus is cleaved at the time of KRS secretion, and this procedure is an important key point in the KRS secretion.

### Example 3

### Binding of Truncated KRS and Syntenin-1

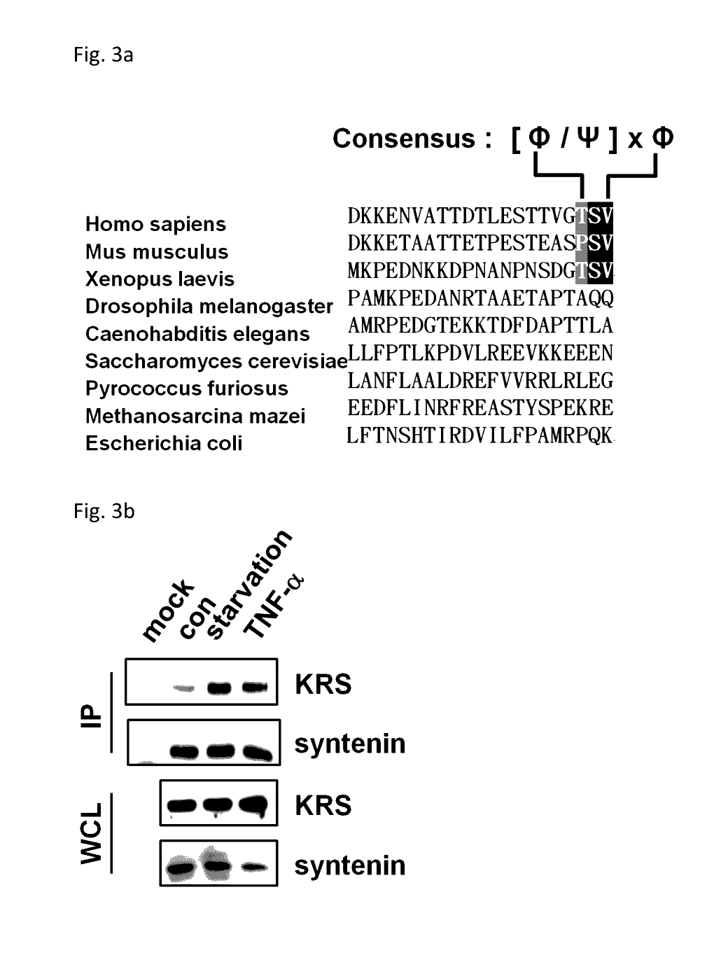

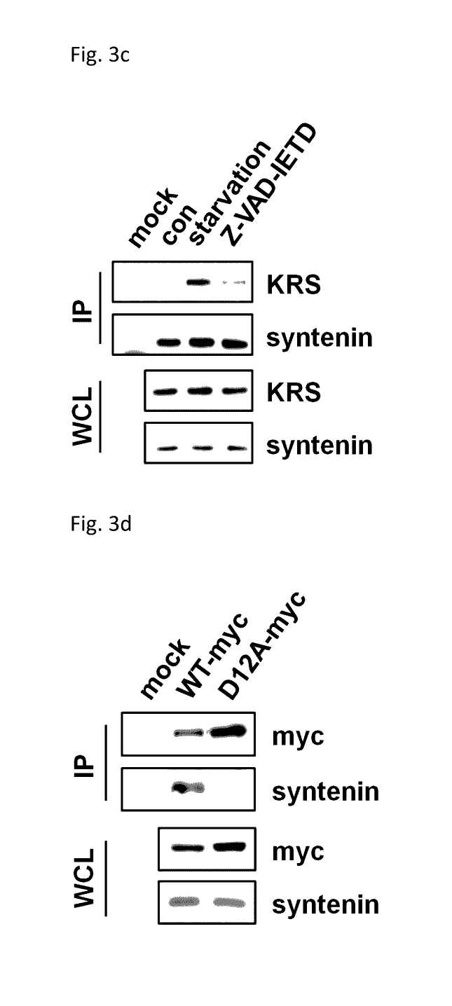

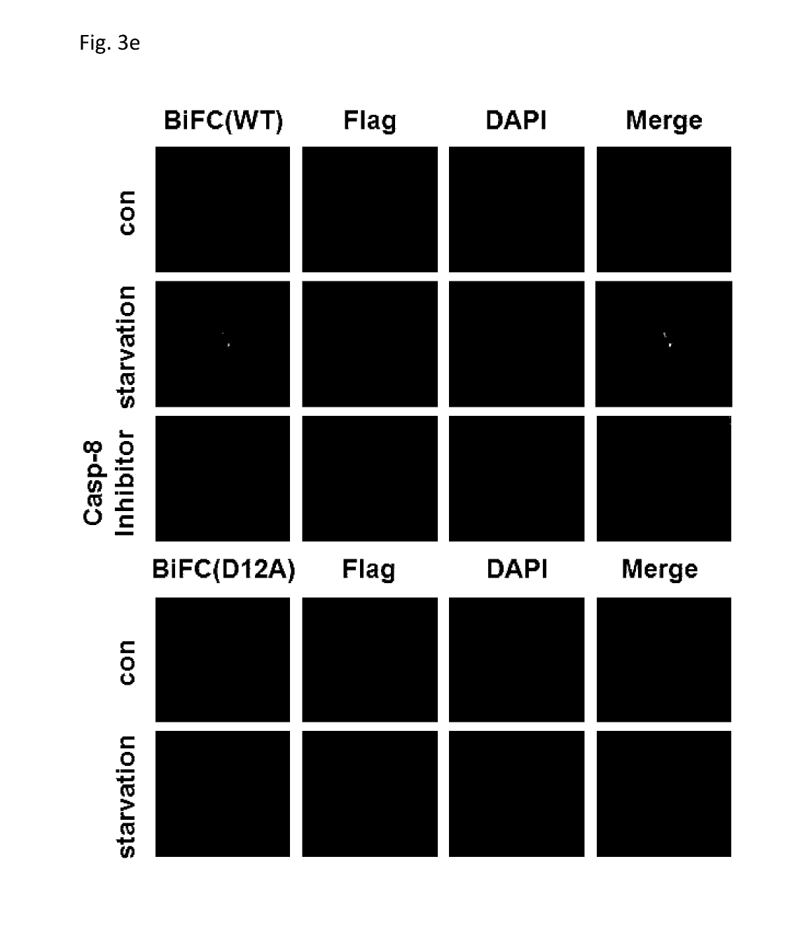

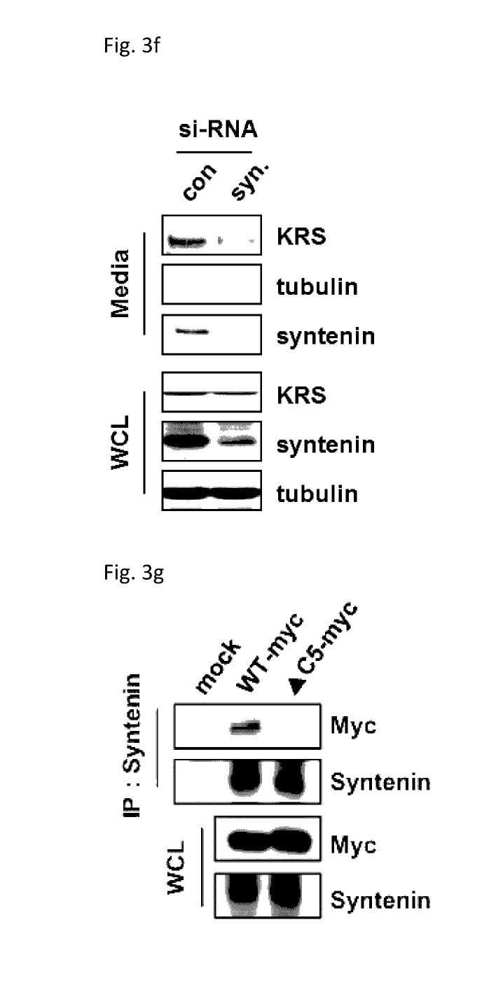

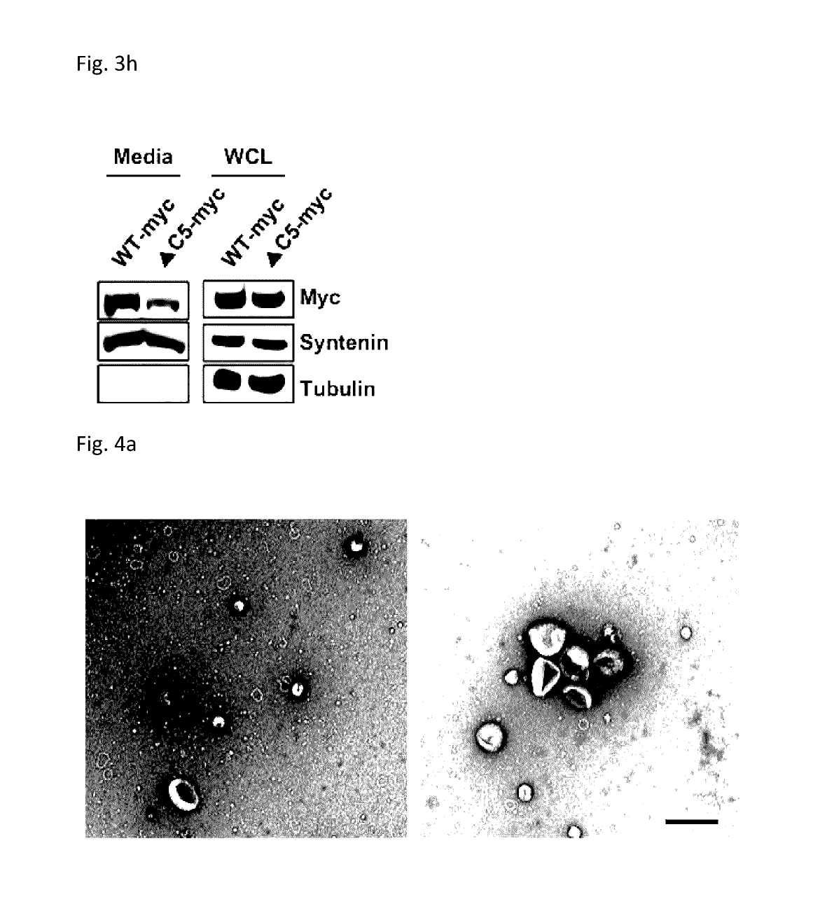

In order to find an answer to the question why the cleavage of KRS by caspase-8 is important in KRS secretion, cytokine activity of KRS was measured. The reason is that the cytokine activity depends on the cleavage for a protein, such as IL1-beta. Upon testing, it was found that the cytokine activity was identical in WT and ▴N12 mutant (13-597 a.a) of KRS. Next, it was assumed that the cleavage of KRS would influence the binding affinity between KRS and another protein. Previous literatures already validated that KRS can bind to syntenin-1. Syntenin-1 is a trafficking protein that functions to move proteins bound thereto inside cells. It has recently been reported that syntenin-1 plays an important role in exosome biogenesis. KRS binds to syntenin-1 through the particular C-terminal sequence thereof. The C-terminus of KRS may be covered by its N-terminus due to its structure characteristics. Therefore, the N-terminus-truncated KRS exposes a larger area of the sequence that binds to syntenin-1, thereby increasing the binding affinity with syntenin. The multiple-alignment confirmed that this portion is also conserved in higher eukaryotes like in the portion cleaved by caspase-8 (see FIG. 3a). In addition, it was verified that the binding of KRS and syntenin-1 was increased by starvation and/or TNF-alpha treatment (see FIG. 3b), and the binding between KRS and syntenin increased by starvation was reduced by the treatment with caspase-8 inhibiting agent (see FIG. 3c). The binding amount of KRS with syntenin-1 was less in D12A, which is not cleaved by caspase-8, than WT (see FIG. 3d). In order to investigate this fact again, the bimolecular fluorescence complementation (BiFC) assay was used. According to the BiFC assay, KRS-vn173 and syntenin-vc155 plasmids, in which the venus proteins cleaved in half bind to KRS and syntenin, respectively, emit the venus (green) fluorescence light only when KRS binds with syntenin. As a result, the BiFC fluorescence was observed at the time of starvation, and the BiFC fluorescence was not observed upon the caspase-8 inhibitor treatment and the use of D12A (see FIG. 3e). The above results confirmed that the cleavage of KRS increased the KRS-syntenin binding. The test regarding the effect of syntenin, which has an increased binding with KRS, on KRS secretion was conducted. As a result of reducing the syntenin protein using si-syntenin, the KRS secretion was definitely reduced compared with the non-reduction of syntenin (see FIG. 3f). In addition, in order to investigate whether the C-terminus of KRS is important in binding with syntenin, deletion mutant KRS with truncated C-terminus (corresponding to 1-592 a.a of the full-length sequence of SEQ ID NO: 2, also designated by ▴c5) was constructed to investigate the binding with syntenin. It was verified that the ability of deletion mutant (1-592 a.a, ▴c5) to bind with syntenin was significantly deteriorated compared with WT (see FIG. 3g). The degree of secretion was investigated using KRS WT and KRS deletion mutant (1-592 a.a, ▴c5) that cannot bind with syntenin. As a result of verification, the amount of secretion was less in the deletion mutant (▴c5) than WT (see FIG. 3h). These results established the fact that the truncation of the N-terminus exposed the syntenin binding motif at the C-terminus of KRS, thereby increasing the binding between syntenin and KRS, and validated that the increased binding with syntenin is important in the KRS secretion.

### Example 4

### Secretion of Truncated KRS Through Exosome Secretion Pathway

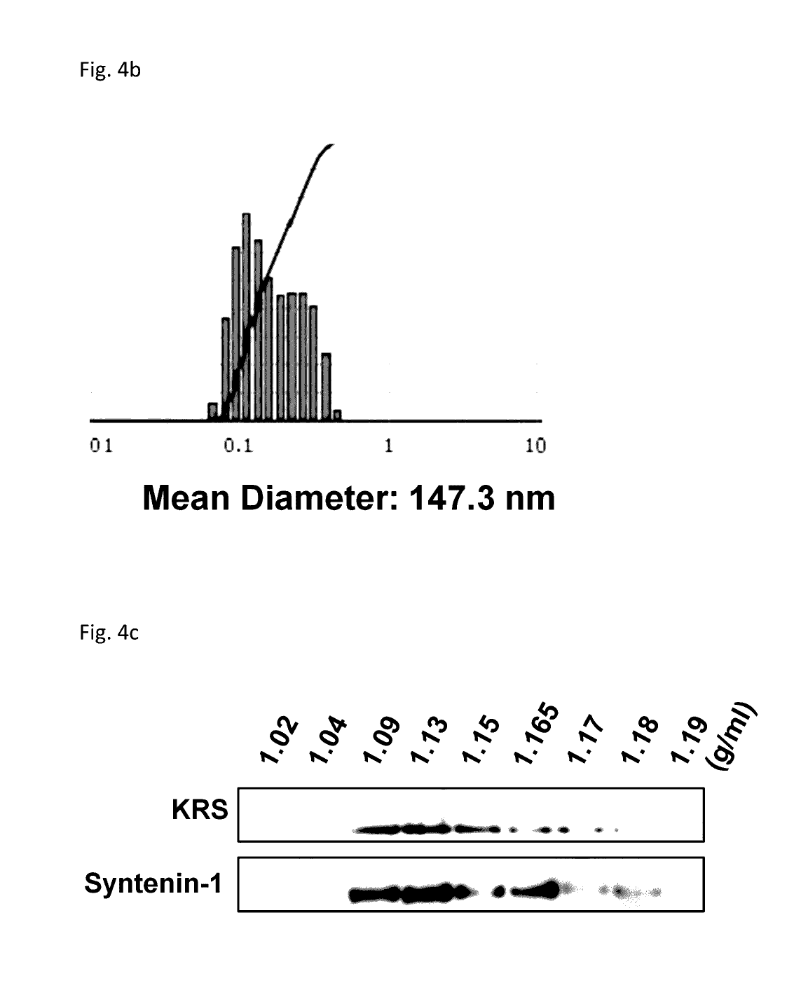

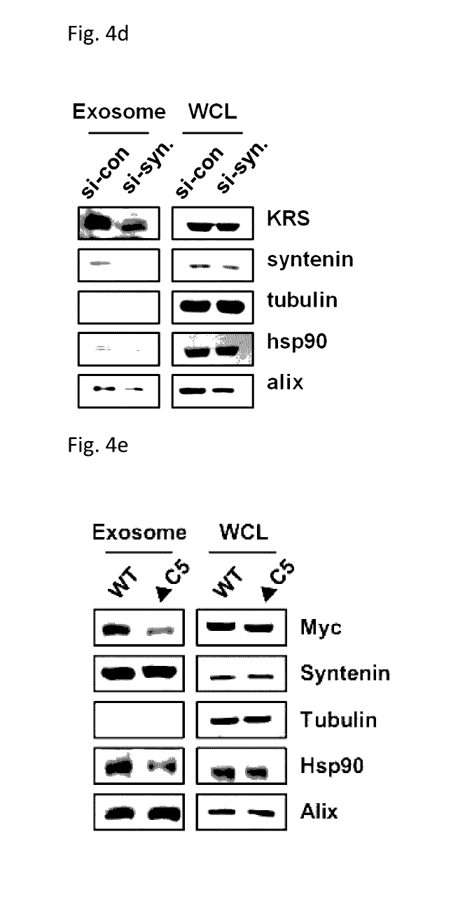

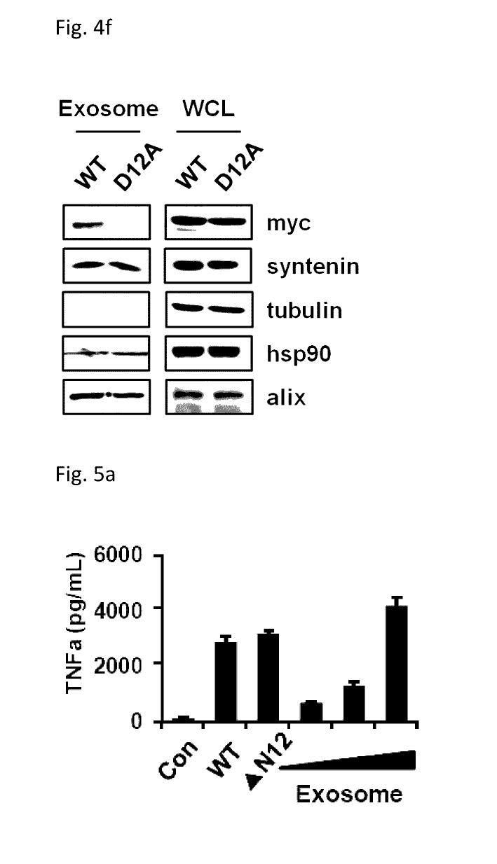

In order to investigate the extracellular secretion of KRS, only vesicles are isolated from KRS-secreted media, followed by electron microscopy analysis. As a result of electron microscopy analysis, cup-shaped figurations are shown (see FIG. 4a). This shape corresponds to the morphology of exosomes. Exosomes have a cup shape in electron microscopy analysis, and are characterized by having a diameter of 50-150 nm and a density of 1.15-1.19 g/ml. The mean diameter of the isolated vesicles is 147.3 nm, which is also consistent to the characteristics of exosomes (see FIG. 4b). Lastly, when the density was investigated using opti-prep gradient assay, KRS-detected vesicles have a density of approximately 1.09-1.15 g/ml. It was also verified that syntenin is present in the same vesicles (see FIG. 4c). These results validated that KRS was secreted together with syntenin into exosomes. In order to investigate the relationship between KRS exosome secretion and syntenin, KRS exosome secretion was investigated after si-syntenin treatment. As a result, the KRS secretion through exosomes was reduced at the time of si-syntenin treatment (see FIG. 4d). As a result of investigating the exosome secretion using deletion mutant (▴c5, 1-592 a.a) that does not bind with syntenin, the exosome secretion was reduced in deletion mutant (▴c5, 1-592 a.a) in comparison with than WT (see FIG. 4e). It was seen through the above two tests that syntenin does not exist together with KRS in the same exosomes, but is involved in KRS exosome secretion. As shown in FIGS. 3a to 3h, it was validated that the truncation of KRS increased the binding of KRS with syntenin. So, lastly, the exosome secretion of untruncated D12A mutant was compared with that of KRS WT. The secretion levels were significantly reduced in D12A mutant compared with KRS WT (see FIG. 4f). To summarize the results, it can be seen that syntenin is important in KRS exosome secretion, and the truncation of KRS plays an essential role in KRS exosome secretion through syntenin.

### Example 5

### Enhancement of KRS Exosome Activity by KRS Truncation

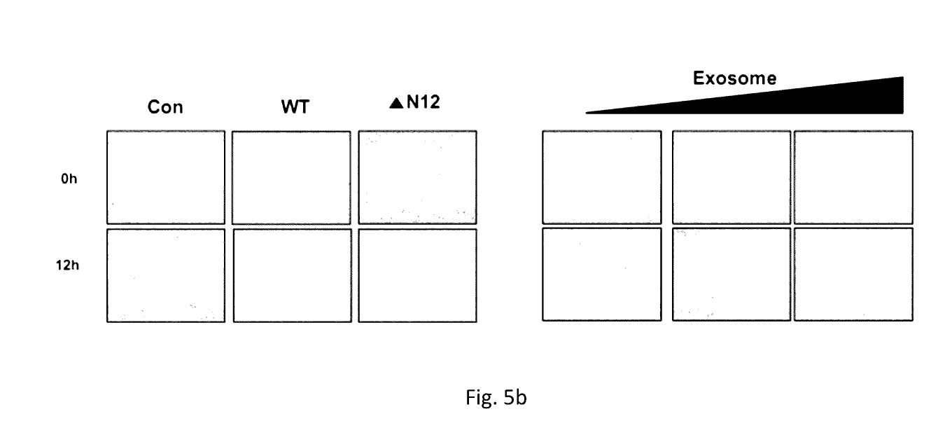

The present inventors investigated that truncated KRS was secreted through exosomes. The exosome is known to be a mediator for cell-cell signaling. The Exosome has various pieces of information with respect to proteins, mi-RNA, tRNA, and the like. Due to these characteristics of the exosome, the information is transferred from a cell to another cell, and due to the transferred information, the cell plays roles different from its original roles. In order to investigate functions of KRS exosomes, KRS WT, truncated KRS (▴N12(13-597 a.a)), and KRS exosomes, respectively, were treated with macrophage for 5 hr to investigate TNF-alpha secretion effects thereof. As a result, it was verified that KRS exosomes have an effect of increasing TNF-alpha secretion, like KRS proteins (see FIG. 5a). These results validated that KRS proteins and KRS exosomes have the same effects. In order to validate this fact again, the increase of migration was investigated using macrophages. The wound-healing assay results verified that the migration of the macrophages was increased 12 hr after the treatment with KRS proteins (WT, ▴N12(13-597 a.a) each) and KRS exosomes (see FIG. 5b). KRS exosomes showed the same effect as in the KRS proteins. To sum the results of the above two tests, it was validated that the KRS fragments contained in the KRS exosome are involved in the activity of KRS exosomes, indicating that the truncation of KRS is important in the activity of KRS exosomes. Therefore, it is believed that the KRS truncated by caspase-8 enhances its binding with syntenin to increase the migration into the exosomes, thereby enhancing the activity of KRS exosomes.

### Example 6

### Cancer Metastasis Effect of ▴N12 KRS and Secretary Exosomes Containing the Same

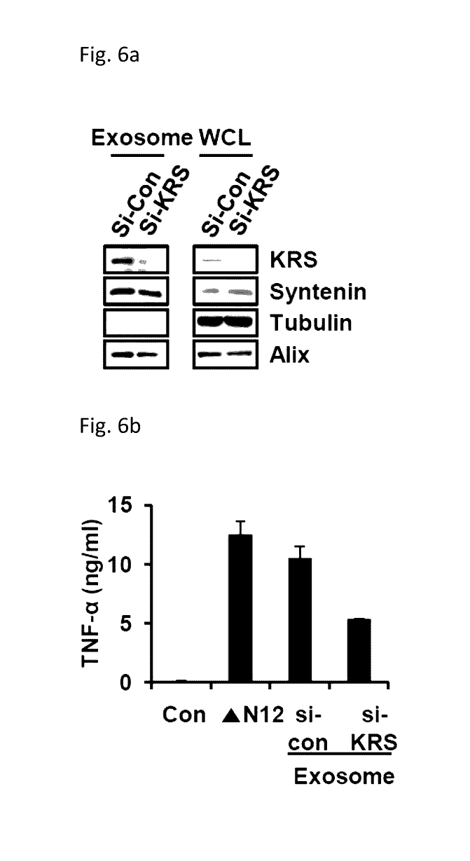

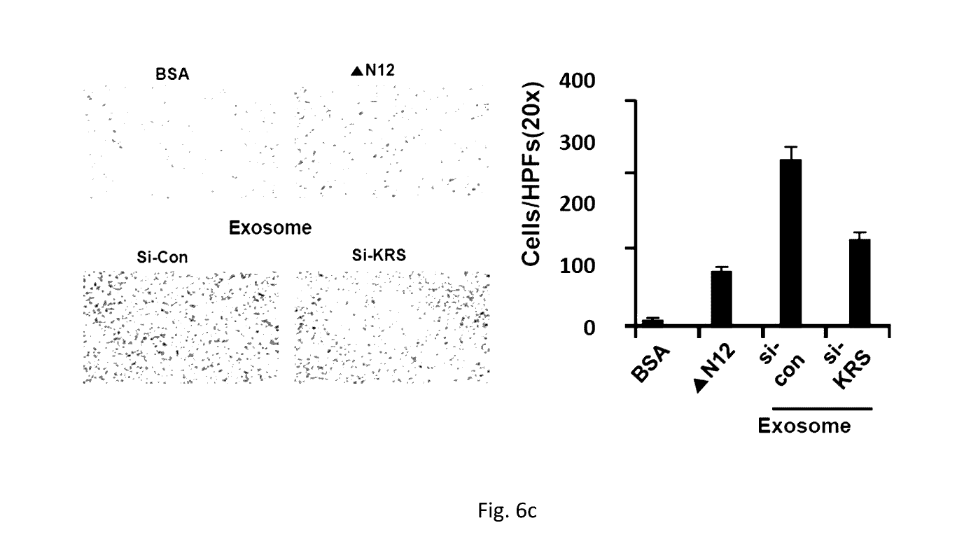

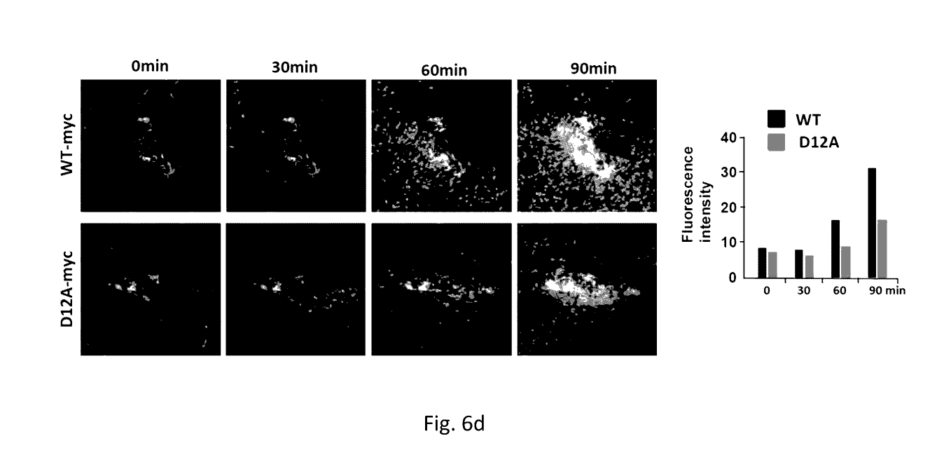

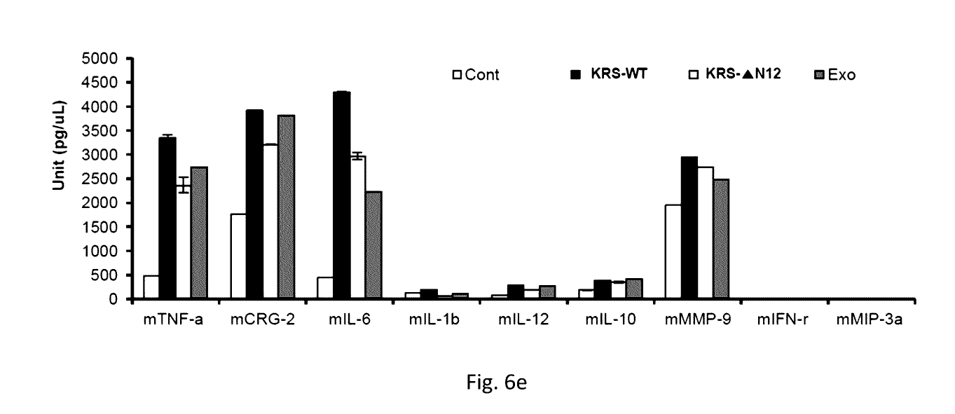

In order to investigate the importance of KRS in inflammation effects due to exosomes as shown in FIG. 5, cells were first treated with si-RNAs (si-con and si-KRS). At 48 hr after si-RNA treatment, the cells were incubated in serum starvation media for 12 hr. The exosomes were purified in the media, and then proteins existing in each exosome were analyzed (hereinafter, the exosome purified in media of si-con treated cells is named si-con exosome; and the exosome purified in media of si-KRS treated cells is named si-KRS exosome, for convenience). As a result of analysis, it was verified that KRS proteins existing in the exosome was reduced in the exosome purified in si-KRS treated cells (FIG. 6a).

In order to investigate the reduction of the exosome inflammation effect by the reduced levels of KRS proteins, the TNF-alpha secretion and the migration effect in macrophages were investigated (FIG. 6b and FIG. 6c). As a result of verification, it can be seen that, when macrophages were treated with si-KRS exosomes, the TNF-alpha secretion from macrophages was reduced and the migration effect was reduced (FIG. 6b and FIG. 6c). Therefore, KRS is an important part of the exosome inflammation activity.

In order to investigate effects of KRS, which is important in the exosome effect, in actual cells and animal models, the intravital confocal visualization of macrophage and neutrophil recruitment test was performed. For the test, B16F10 cell (Mus musculus skin melanoma) was first used. Since immune responses due to a difference in species occur in the mouse test using general human cancer cells, B16F10 cell was used. KRS WT-myc and KRS D12A-myc were transfected in B16F10 cells, followed by incubation for 24 hr. After 24 hr, the respective cells were injected at 4×10⁴ into the back skin of the ears in LysM-GFP (Lysozyme M-GFP) mice (mice having GFP-expressed macrophages and neutrophils). After the injection, the recruitment of macrophages and neutrophils was verified during the time course. The recruitment of, at the maximum, twice as many macrophages and neutrophils was confirmed when KRS WT-myc was injected into the transgenic cells than when KRS D12A-myc was injected (FIG. 6d). These results again established the fact that KRS is important in cancer related inflammation through exosomes.

In order to investigate accurate mechanisms of inflammation by KRS, various inflammatory cytokine secretion types were investigated using KRS proteins (KRS-WT, KRS▴N12) and KRS exosomes. As shown in FIG. 6e, it was found that KRS proteins and KRS exosomes induced the secretion of IL-6, mCRG-2, and MMP9 as well as TNF-alpha from macrophages. Mouse CRG-2 is a protein pertaining to cxc chemokines, and functions to enhance the macrophage recruitment. It has been reported that a large number of macrophages are associated with cancer metastasis and poor prognosis in actual tumor microenvironments. IL-6 acts on cancer cells to lower E-cadherin. The reduction of E-cadherin is one of the important procedures of cancer metastasis, and is one of the important markers for the epithelial-mesenchymal transition (EMT) mechanism that increases cancer cell motility. MMP9 plays an important role in extracellular matrix degradation and vascular remodeling. Therefore, it can be seen that the cytokines secreted from macrophages by KRS-expressed exosomes (that is, KRS exosomes) help the metastasis of all cancer cells. That is, the inflammation reaction occurring by KRS-expressed exosomes is anticipated to help the cancer cell metastasis, and KRS having an important role in exosome activity is thought to play an important role in cancer metastasis.

### INDUSTRIAL APPLICABILITY

As set forth above, the present invention is directed to a lysyl tRNA synthetase (KRS) fragment comprising the amino acid sequence represented by SEQ ID NO: 1 and being secreted from cancer cells, microvesicles containing the KRS fragment, and methods for providing information necessary for the diagnosis of cancer and screening a cancer metastasis inhibiting agent using the same. The present invention can be favorably used in the development of a diagnostic kit for providing information necessary for diagnosis of cancer or the development of a cancer metastasis inhibiting agent, and thus the present invention is highly industrially applicable.

1. A lysyl tRNA synthetase fragment comprising an amino acid sequence represented by SEQ ID NO: 1 and being secreted from cancer cells.

2. Microvesicles comprising the lysyl tRNA synthetase fragment of claim 1 and being secreted from cancer cells.

3. The microvesicles of claim 2, wherein the microvesicles are exosomes.

4. The microvesicles of claim 2, wherein the cancer is at least one selected from the group consisting of breast cancer, colorectal cancer, lung cancer, small cell lung cancer, gastric cancer, liver cancer, blood cancer, bone cancer, pancreatic cancer, skin cancer, head or neck cancer, cutaneous or intraocular melanoma, uterine cancer, ovarian cancer, rectal cancer, anal cancer, colon cancer, fallopian tube carcinoma, endometrial carcinoma, cervical cancer, vaginal cancer, vulvar carcinoma, Hodgkin's disease, esophageal cancer, small intestine cancer, endocrine cancer, thyroid cancer, parathyroid carcinoma, adrenal cancer, soft tissue sarcoma, urethral cancer, penile cancer, prostate cancer, chronic or acute leukemia, lymphocyte lymphoma, bladder cancer, kidney cancer, ureter cancer, renal cell carcinoma, renal pelvic carcinoma, CNS tumor, primary CNS lymphoma, spinal cord tumor, brain stem glioma, and pituitary adenoma.

5. A method for providing information necessary for the diagnosis of cancer, the method comprising:
(a) isolating microvesicles from a biological sample taken from a suspected cancer subject;
(b) disrupting the microvesicles in step (a) to measure the level of the lysyl tRNA synthetase fragment of claim 1 or the expression level of a gene encoding the fragment; and
(c) comparing the level of the fragment or the expression level of the gene encoding the fragment with the level of the fragment or the expression level of the gene encoding the fragment in a normal control sample.

6. A method for screening a cancer metastasis inhibiting agent, the method comprising:
(A) bringing a lysyl tRNA synthetase (KRS) or a KRS fragment comprising the C-terminal region thereof, syntenin, and a test agent into contact with one another;
(B) measuring a change in the binding level of the KRS or fragment thereof and syntenin; and
(C) bringing the test agent, which is determined to change the binding level of the KRS or fragment thereof and syntenin, into contact with cancer cells, to evaluate whether the microvesicles of claim 2 are secreted from the cancer cells.

7. The method of claim 6, wherein the C-terminal region of the lysyl tRNA synthetase (KRS) in step (A) consists of the amino acid sequence of SEQ ID NO: 6.

8. The method of claim 6, wherein the KRS fragment in step (A) consists of the amino acid sequence of SEQ ID NO: 1.

9. The method of claim 6, wherein the syntenin in step (A) is a polypeptide comprising the amino acid sequence of SEQ ID NO: 4.

10. The method of claim 6, the method further comprises (D) administering the test agent, which is determined to inhibit the secretion of the microvesicles of claim 2 in step (C), to an animal with cancer, to evaluate whether the test agent exhibits an effect of preventing or treating cancer metastasis.


SEQUENCE LISTING (6 sequences)

SEQ ID NO: 1 - protein, 585 aa, Artificial Sequence
  Truncated KRS (13-597a.a)
GSEPKLSKNELKRRLKAEKKVAEKEAKQKELSEKQLSQATAAATNHTTDNGVGPEEESVDPNQYYKIRSQAIHQLKVNGEDPYPHKFHVDISLTDFIQKYSHLQPGDHLTDITLKVAGRIHAKRASGGKLIFYDLRGEGVKLQVMANSRNYKSEEEFIHINNKLRRGDIIGVQGNPGKTKKGELSIIPYEITLLSPCLHMLPHLHFGLKDKETRYRQRYLDLILNDFVRQKFIIRSKIITYIRSFLDELGFLEIETPMMNIIPGGAVAKPFITYHNELDMNLYMRIAPELYHKMLVVGGIDRVYEIGRQFRNEGIDLTHNPEFTTCEFYMAYADYHDLMEITEKMVSGMVKHITGSYKVTYHPDGPEGQAYDVDFTPPFRRINMVEELEKALGMKLPETNLFETEETRKILDDICVAKAVECPPPRTTARLLDKLVGEFLEVTCINPTFICDHPQIMSPLAKWHRSKEGLTERFELFVMKKEICNAYTELNDPMRQRQLFEEQAKAKAAGDDEAMFIDENFCTALEYGLPPTAGWGMGIDRVAMFLTDSNNIKEVLLFPAMKPEDKKENVATTDTLESTTVGTSV

SEQ ID NO: 2 - protein, 597 aa, Artificial Sequence
  KRS (Genbank Accession No. NP_005539.1)
MAAVQAAEVKVDGSEPKLSKNELKRRLKAEKKVAEKEAKQKELSEKQLSQATAAATNHTTDNGVGPEEESVDPNQYYKIRSQAIHQLKVNGEDPYPHKFHVDISLTDFIQKYSHLQPGDHLTDITLKVAGRIHAKRASGGKLIFYDLRGEGVKLQVMANSRNYKSEEEFIHINNKLRRGDIIGVQGNPGKTKKGELSIIPYEITLLSPCLHMLPHLHFGLKDKETRYRQRYLDLILNDFVRQKFIIRSKIITYIRSFLDELGFLEIETPMMNIIPGGAVAKPFITYHNELDMNLYMRIAPELYHKMLVVGGIDRVYEIGRQFRNEGIDLTHNPEFTTCEFYMAYADYHDLMEITEKMVSGMVKHITGSYKVTYHPDGPEGQAYDVDFTPPFRRINMVEELEKALGMKLPETNLFETEETRKILDDICVAKAVECPPPRTTARLLDKLVGEFLEVTCINPTFICDHPQIMSPLAKWHRSKEGLTERFELFVMKKEICNAYTELNDPMRQRQLFEEQAKAKAAGDDEAMFIDENFCTALEYGLPPTAGWGMGIDRVAMFLTDSNNIKEVLLFPAMKPEDKKENVATTDTLESTTVGTSV

SEQ ID NO: 3 - protein, 625 aa, Artificial Sequence
  KRS isoform(Genbank Accession No. NP_001123561.1)
MLTQAAVRLVRGSLRKTSWAEWGHRELRLGQLAPFTAPHKDKSFSDQRSELKRRLKAEKKVAEKEAKQKELSEKQLSQATAAATNHTTDNGVGPEEESVDPNQYYKIRSQAIHQLKVNGEDPYPHKFHVDISLTDFIQKYSHLQPGDHLTDITLKVAGRIHAKRASGGKLIFYDLRGEGVKLQVMANSRNYKSEEEFIHINNKLRRGDIIGVQGNPGKTKKGELSIIPYEITLLSPCLHMLPHLHFGLKDKETRYRQRYLDLILNDFVRQKFIIRSKIITYIRSFLDELGFLEIETPMMNIIPGGAVAKPFITYHNELDMNLYMRIAPELYHKMLVVGGIDRVYEIGRQFRNEGIDLTHNPEFTTCEFYMAYADYHDLMEITEKMVSGMVKHITGSYKVTYHPDGPEGQAYDVDFTPPFRRINMVEELEKALGMKLPETNLFETEETRKILDDICVAKAVECPPPRTTARLLDKLVGEFLEVTCINPTFICDHPQIMSPLAKWHRSKEGLTERFELFVMKKEICNAYTELNDPMRQRQLFEEQAKAKAAGDDEAMFIDENFCTALEYGLPPTAGWGMGIDRVAMFLTDSNNIKEVLLFPAMKPEDKKENVATTDTLESTTVGTSV

SEQ ID NO: 4 - protein, 298 aa, Artificial Sequence
  Syntenin-1
MSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQGIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAFIFEHIIKRMAPSIMKSLMDHTIPEV

SEQ ID NO: 5 - DNA, 1758 bp, Artificial Sequence
  DNA sequence for truncated KRS (13-597a.a)
ggcagcgagccgaaactgagcaagaatgagctgaagagacgcctgaaagctgagaagaaagtagcagagaaggaggccaaacagaaagagctcagtgagaaacagctaagccaagccactgctgctgccaccaaccacaccactgataatggtgtgggtcctgaggaagagagcgtggacccaaatcaatactacaaaatccgcagtcaagcaattcatcagctgaaggtcaatggggaagacccatacccacacaagttccatgtagacatctcactcactgacttcatccaaaaatatagtcacctgcagcctggggatcacctgactgacatcaccttaaaggtggcaggtaggatccatgccaaaagagcttctgggggaaagctcatcttctatgatcttcgaggagagggggtgaagttgcaagtcatggccaattccagaaattataaatcagaagaagaatttattcatattaataacaaactgcgtcggggagacataattggagttcaggggaatcctggtaaaaccaagaagggtgagctgagcatcattccgtatgagatcacactgctgtctccctgtttgcatatgttacctcatcttcactttggcctcaaagacaaggaaacaaggtatcgccagagatacttggacttgatcctgaatgactttgtgaggcagaaatttatcatccgctctaagatcatcacatatataagaagtttcttagatgagctgggattcctagagattgaaactcccatgatgaacatcatcccagggggagccgtggccaagcctttcatcacttatcacaacgagctggacatgaacttatatatgagaattgctccagaactctatcataagatgcttgtggttggtggcatcgaccgggtttatgaaattggacgccagttccggaatgaggggattgatttgacgcacaatcctgagttcaccacctgtgagttctacatggcctatgcagactatcacgatctcatggaaatcacggagaagatggtttcagggatggtgaagcatattacaggcagttacaaggtcacctaccacccagatggcccagagggccaagcctacgatgttgacttcaccccacccttccggcgaatcaacatggtagaagagcttgagaaagccctggggatgaagctgccagaaacgaacctctttgaaactgaagaaactcgcaaaattcttgatgatatctgtgtggcaaaagctgttgaatgccctccacctcggaccacagccaggctccttgacaagcttgttggggagttcctggaagtgacttgcatcaatcctacattcatctgtgatcacccacagataatgagccctttggctaaatggcaccgctctaaagagggtctgactgagcgctttgagctgtttgtcatgaagaaagagatatgcaatgcgtatactgagctgaatgatcccatgcggcagcggcagctttttgaagaacaggccaaggccaaggctgcaggtgatgatgaggccatgttcatagatgaaaacttctgtactgccctggaatatgggctgccccccacagctggctggggcatgggcattgatcgagtcgccatgtttctcacggactccaacaacatcaaggaagtacttctgtttcctgccatgaaacccgaagacaagaaggagaatgtagcaaccactgatacactggaaagcacaacagttggcacttctgtctag

SEQ ID NO: 6 - protein, 5 aa, Artificial Sequence
  KRS-C-terminal
VGTSV